(19) United States
(12) Patent Application Publication
(10) Pub. No.: US 2025/0127606 A1
(43) Pub. Date: Apr. 24, 2025

(54) SYSTEMS AND METHODS FOR PERFORMING ENDOSCOPIC PROCEDURES

(71) Applicant: BOSTON SCIENTIFIC SCIMED, INC., Maple Grove, MN (US)

(72) Inventors: Saurav V. Gupta, Medway, MA (US); Camron Hagemeyer, Bloomington, IN (US); Marc A. Barthet, Marseille (FR); Jean-Michel Gonzalez, Marseille (FR); Barry Weitnzer, Acton, MA (US); Dan Bacon, Fitchburg, MA (US); Kenneth R. Keene, Winchester, MA (US); John B. Golden, Norton, MA (US); Jeffrey V. Bean, Fitchburg, MA (US)

(73) Assignee: BOSTON SCIENTIFIC SCIMED, INC., Maple Grove, MN (US)

(21) Appl. No.: 19/005,848

(22) Filed: Dec. 30, 2024

Related U.S. Application Data
(63) Continuation of application No. 16/361,733, filed on Mar. 22, 2019.
(60) Provisional application No. 62/650,075, filed on Mar. 29, 2018.
(60) Provisional application No. 62/650,080, filed on Mar. 29, 2018.

(51) Int. Cl. A61F 2/04; A61B 1/018; A61B 1/273; A61B 5/06; A61B 17/00; A61B 17/11; A61B 17/12; A61B 17/29; A61B 17/30; A61B 90/00; A61B 90/30; A61F 5/00; A61M 25/01

(52) U.S. Cl. CPC A61F 2/04; A61B 1/018; A61B 1/2733; A61B 1/2736; A61B 5/061; A61B 17/1114; A61B 17/29; A61B 90/30; A61F 5/0079; A61F 5/0089; A61B 2017/00221; A61B 2017/00278; A61B 2017/00336; A61B 2017/00818; A61B 2017/00862; A61B 2017/00867; A61B 2017/1125; A61B 2017/1139; A61B 17/12031; A61B 17/12099; A61B 17/12172; A61B 2017/2924; A61B 2017/2944; A61B 2017/305; A61B 2090/0811; A61B 2090/306; A61B 2090/309; A61B 2090/3945; A61F 2002/045; A61F 2220/0008; A61F 2230/0095; A61F 2250/0059; A61M 2025/0166

(57) ABSTRACT

According to exemplary embodiments of the present disclosure, devices, systems, and methods for endoscopic procedures may include an end effector of a grasping system having a first arm and a second arm. The first and second arms may extend from a common proximal joint. The first and second arms may be formed of a self-expanding material and may have a spring force to set the arms in an unconstrained open position. The first and second arms may have an arc shape such that a distal tip of each first and second arm are configured to close as the first and second arms are actuated to a constrained closed position. A catheter may include a locating element at a distal end of a flexible tube. The locating element may be configured for locating the grasping system.

### CROSS-REFERENCE TO RELATED APPLICATIONS

This application is a continuation of and claims the benefit of the earlier filing date of U.S. patent application Ser. No. 16/361,733, filed Mar. 22, 2019, which claims the benefit of priority under 35 U.S.C. § 119 of U.S. Provisional Application Ser. No. 62/650,075, filed Mar. 29, 2018, and to U.S. Provisional Application Ser. No. 62/650,080, filed Mar. 29, 2018, and which applications are incorporated herein by reference in their entireties for all purposes. Any and all priority claims identified in the Application Data Sheet, or any correction thereto, are hereby incorporated by reference under 37 C.F.R. § 1.57.

### FIELD

The present disclosure relates generally to systems and methods for performing endoscopic procedures, and, more particularly, to location and access devices, systems, and methods for gastrojejunostomy procedures.

### BACKGROUND

Obesity affects a growing population and may cause additional diseases such as type 2 diabetes, greatly increasing risk of a patient's health. Surgical procedures such as bariatric surgery, e.g., to restrict a portion of a stomach and/or bypass portions of the intestine, may be the only option for patients categorized as morbidly obese. Additionally, these types of procedures may have significant side effects such as enteric hormonal changes, and are relatively invasive surgical procedures with associated complications, tissue trauma, and/or infections, which in some instances may put the patient at risk.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, an end effector of a grasping system for an endoscopic procedure may include a first arm and a second arm. The first and second arms may extend from a common proximal joint, and the first and second arms may be formed of a self-expanding material and may have a spring force to set the arms in an unconstrained open position. The first and second arms may have an arc shape such that a distal tip of each first and second arm may be configured to close as the first and second arms are actuated to a constrained closed position.

In various of the foregoing and other embodiments of the present disclosure, the first and second arms may include one or more projections on an inner surface. The one or more projections may be castellations. The castellations may be in the form of one or a combination of: a series of equally spaced projections and grooves forming atraumatic jaws of the first and second arms; a series of projections as rounded teeth sized and shaped differently from grooves forming alligator jaws of the first and second arms; a series of projections and grooves forming trapezoidal shapes; or a series of projections and grooves including a projection having a curvature forming a hook in the projections. The common proximal joint may be a single connection of the first and second arms from a unitary proximal end. The end effector may be actuatable relative to a sheath, such that the first and second arms may be actuated between an unconstrained open position in response to the end effector extending out of the sheath, and a constrained closed position in response to the end effector retracting within the sheath. In the constrained closed position, the arc shape of the first and second arms may form an oval shape. The projections on the inner surfaces of the first and second arms may provide atraumatic grasping of tissue, the tissue including a body lumen.

According to an exemplary embodiment of the present disclosure, a system for an endoscopic procedure may include a grasping device. The grasping device may include an end effector disposed at a distal end of the grasping system including a first arm and a second arm. The first and second arms may extend from a single connection at a proximal end, and the first and second arms may be formed of a self-expanding material and may have a spring force to set the arms in an unconstrained open position. The grasping device may further include an end effector disposed at a distal end of the grasping system. The end effector may include a first arm and a second arm. The first and second arms may extend from a single connection at a proximal end, and the first and second arms may be formed of a self-expanding material and may have a spring force to set the arms in an unconstrained open position. The grasping device may further include a handle disposed at a proximal end of the grasping system. The handle may include a movable portion for actuating the first and second arms of the end effector between an unconstrained open position and a constrained closed position. The system may further include a drive wire connecting the end effector and the handle, and a catheter may include a locating element at a distal end of a flexible tube.

In various of the foregoing and other embodiments of the present disclosure, the first and second arms may have an arc shape such that a distal tip of each first and second arm may be configured to close as the first and second arms are actuated to a constrained closed position. The first and second arms may include one or more projections on an inner surface. The one or more projections may be castellations. The castellations may be in the form of one or a combination of: a series of equally spaced projections and grooves forming atraumatic jaws of the first and second arms; a series of projections as rounded teeth sized and shaped differently from grooves forming alligator jaws of the first and second arms; a series of projections and grooves forming trapezoidal shapes; or a series of projections and grooves including a projection having a curvature forming a hook in the projections. The end effector may be actuatable relative to a sheath, such that the first and second arms may be actuated between the unconstrained open position in response to the end effector extending out of the sheath, and the constrained closed position in response to the end effector retracting within the sheath. The catheter may include one or more projections for engaging with gastrointestinal tissue during peristaltic contractions. The one or more projections of the catheter may include any of the following: a frustoconical section extending radially from a surface of the catheter, the frustoconical section having a planar section and connecting a larger diameter of the frustoconical section to the catheter; a plurality of protrusions extending radially from the surface of the catheter, the plurality of protrusions being equidistantly spaced from each other; or a helical feature having a thread extending radially from the surface of the catheter. The locating element of the catheter may include a light emitting element, a sensor, a transmitter, or a receiver, or combinations thereof. The locating element may be configured for locating the grasping device. The drive wire may be connected to the movable portion of the handle, for actuating the end effector between the unconstrained open position in response to the end effector extending out of the sheath, and the constrained closed position in response to the end effector retracting within the sheath. In various embodiments, a catheter may be a nasocatheter

According to an exemplary embodiment of the present disclosure, a method for performing an endoscopic procedure on a patient may include inserting a catheter in the patient. A distal end of a catheter may be selectively positioned in a small bowel of the patient. The method may further include inserting an endoscope in the patient. A distal end of the endoscope may be selectively positioned in a stomach of the patient based on the selected position of the catheter in the small bowel. The method may further include actuating a grasping system to extend an end effector for grasping selected tissue of the small bowel at the selected position of the catheter. The end effector may include a first arm and a second arm extending from a single connection at a proximal end of the arms, and may have an arc shape such that a distal tip of each first and second arm may be configured for atraumatically closing around the small bowel as the first and second arms are actuated to a closed position.

In various of the foregoing and other embodiments of the present disclosure, the catheter may be selectively positioned at a first location in the small bowel and the endoscope may be selectively positioned at a second location apposing the first location. The method may further include creating an opening in the stomach, and grasping the tissue of the small bowel with the grasping system through the stomach opening. The method may further include creating an opening in the small bowel and delivering an anastomotic device across the openings. The device may appose the stomach and small bowel at the respective selected positions and creates a conduit for stomach content to flow therethrough. The catheter may include projections that may be acted upon by peristaltic motion to propel the catheter to the selected position in the small bowel. The end effector may be actuatable relative to a sheath, such that the first and second arms may be actuated between an unconstrained open position in response to the end effector extending out of the sheath, and a constrained closed position in response to the end effector retracting within the sheath.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a human gastrointestinal system;

FIG. 2A illustrates a passage of a catheter into a patient;

FIG. 2B illustrates a peristaltic movement of a patient's intestinal tract;

FIG. 3 illustrates an exemplary embodiment of a nasocatheter in accordance with the present disclosure;

FIG. 3A illustrates a portion of a tube of an exemplary embodiment of a nasocatheter in accordance with the present disclosure;

FIG. 3B illustrates a light emitting diode configuration of an exemplary embodiment of a nasocatheter in accordance with the present disclosure;

FIG. 3C illustrates a sensor configuration of an exemplary embodiment of a nasocatheter in accordance with the present disclosure;

FIGS. 4A-4C illustrate another exemplary embodiment of a nasocatheter in accordance with the present disclosure;

FIGS. 5A-5C illustrate another exemplary embodiment of a nasocatheter in accordance with the present disclosure;

FIGS. 6A-6C illustrate another exemplary embodiment of a nasocatheter in accordance with the present disclosure;

FIGS. 7A-7C illustrate another exemplary embodiment of a nasocatheter in accordance with the present disclosure;

FIGS. 8A-8C illustrate another exemplary embodiment of a nasocatheter in accordance with the present disclosure;

FIG. 9A illustrates an exemplary embodiment of a grasping system in accordance with the present disclosure;

FIG. 9B illustrates an exemplary embodiment of an end effector of a grasping system in accordance with the present disclosure;

FIG. 9C illustrates an exemplary embodiment of tubing of a grasping system in accordance with the present disclosure;

FIG. 9D illustrates an exemplary embodiment of an end effector of a grasping system while grasping tissue in accordance with the present disclosure;

FIG. 10A illustrates an exemplary embodiment of a distal end of a grasping system in an unconstrained position in accordance with the present disclosure;

FIG. 10B illustrates an exemplary embodiment of a distal end of a grasping system in a constrained position in accordance with the present disclosure;

FIG. 11A illustrates an exemplary embodiment of a distal end of a grasping system by actuation of a drive wire in accordance with the present disclosure;

FIG. 11B illustrates another exemplary embodiment of a distal end of a grasping system by actuation of a sheath in accordance with the present disclosure;

FIG. 12A illustrates an exemplary embodiment of an end effector of a grasping system in accordance with the present disclosure;

FIG. 12B illustrates another exemplary embodiment of an end effector of a grasping system in accordance with the present disclosure;

FIGS. 13A-13E illustrate exemplary embodiments of end effector arms of a grasping system in accordance with the present disclosure;

FIG. 14A illustrates an exemplary embodiment of a grasping system including a handle in accordance with the present disclosure;

FIGS. 14B-14G illustrate exemplary embodiments of a handle of a grasping system in accordance with the present disclosure;

FIGS. 15A-15B illustrate an exemplary embodiment of a handle of a grasping system in accordance with the present disclosure;

FIG. 15C illustrates an exploded view of an exemplary embodiment of a rotator knob of a handle of a grasping system in accordance with the present disclosure; and

FIGS. 16A-16F illustrate an exemplary embodiment of a process for creating an anastomosis in accordance with the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

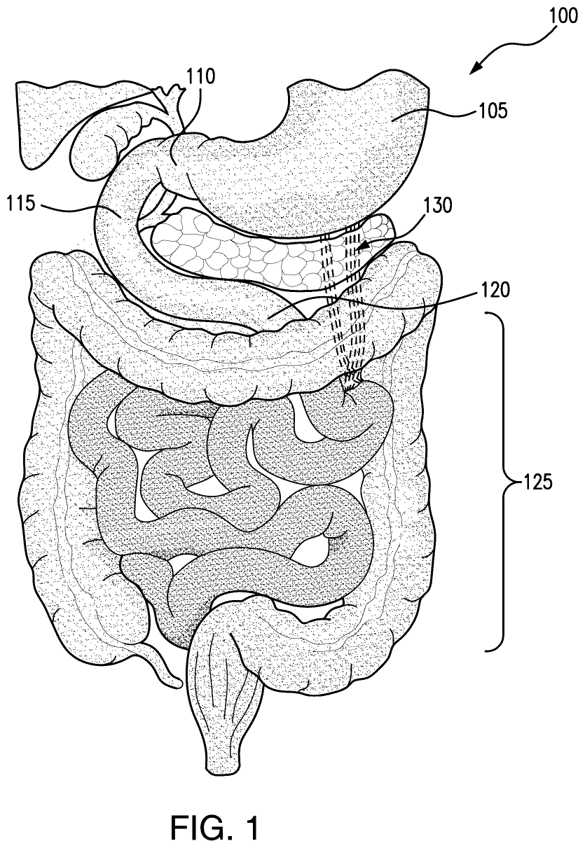

FIG. 1 illustrates a gastrointestinal system of a patient. According to exemplary embodiments of the present disclosure, a natural orifice transluminal endoscopic surgery (NOTES) procedure may be advantageous over other types of bypass procedures from a stomach 105 to a jejunum 120 (e.g., an endoscopic ultrasound procedure) so that a jejunal loop, or a loop of small bowel in the jejunum, may be selected a distance from the pylorus 110, for example, by creating an anastomosis farther into the jejunum 120 that may not otherwise be possible using other systems. In this manner, stomach contents (e.g., food and other nutrients) may not be absorbed as it travels from the stomach 105 through the small bowel 125, promoting patient weight loss and possible controlling type-2 diabetes. For example, the hindgut hypothesis states that diabetes control results from the more rapid delivery of nutrients to the distal small intestine, thereby enhancing the release of hormones such as glucagon-like peptide-1 (GLP-1). The Foregut hypothesis states that exclusion of the proximal small intestine reduces or suppresses the secretion of anti-incretin hormones, leading to improvement of blood glucose control. Thus, type-2 diabetes may be controllable, along with weight loss, by bypassing a longer portion of the jejunum 120, e.g., creating an anastomosis approximately 150 cm or greater from the pylorus 110 at the duodenum 115.

The present disclosure relates to devices, systems, and methods for performing a procedure, e.g., an endoscopic, laparoscopic, and/or open surgical procedure, to create a gastrojejunal anastomosis. For example, devices and systems described herein may aide gastrojejunal anastomosis placement by reliably and repeatably locating a desired position in a patient's gastrointestinal system, e.g., distinguishing a position in the jejunum 120 proximal and/or distal to the Ligament of Treitz 130. Additionally, devices and systems may allow for a physician to better grasp and/or hold a portion of the small bowel 125 during a gastrojejunal anastomosis procedure. Although the systems and devices are described herein with respect to a gastrointestinal system, it may be understood that exemplary embodiments of devices and systems in accordance with the present disclosure may be advantageous for use in any other procedures and/or anatomy, where selective location is blind and atraumatic grasping of tissue (e.g., a body lumen and/or other sensitive tissue structures) is indicated.

It may be understood that references to “proximal” may be defined as an end of the systems and devices closest to the entry point of the patient (e.g., a nasal and/or oral cavity) and “distal” may be defined as an end of the systems and devices closest to the desired location of the system and devices in the patient (e.g., a patient's gastrointestinal system such as the jejunum).

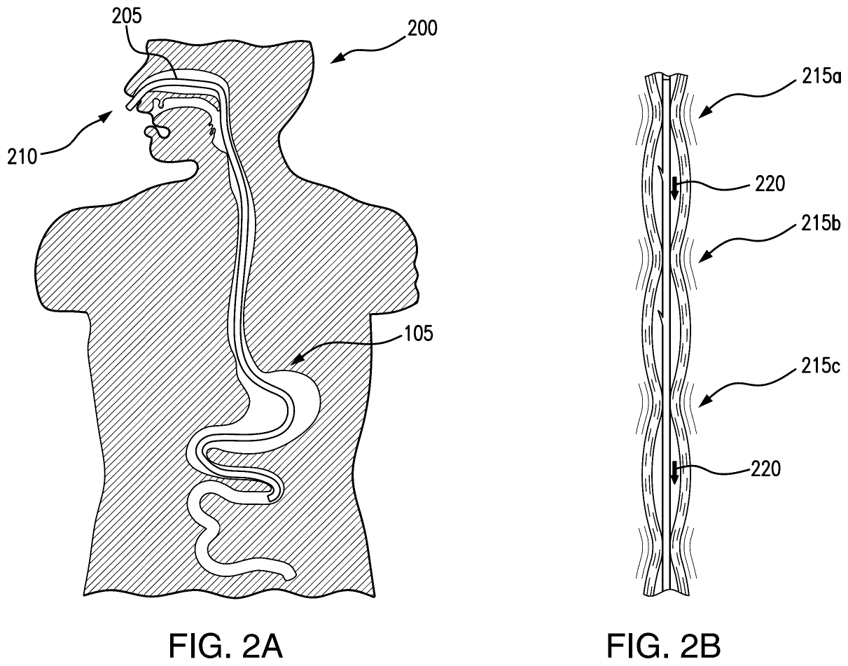

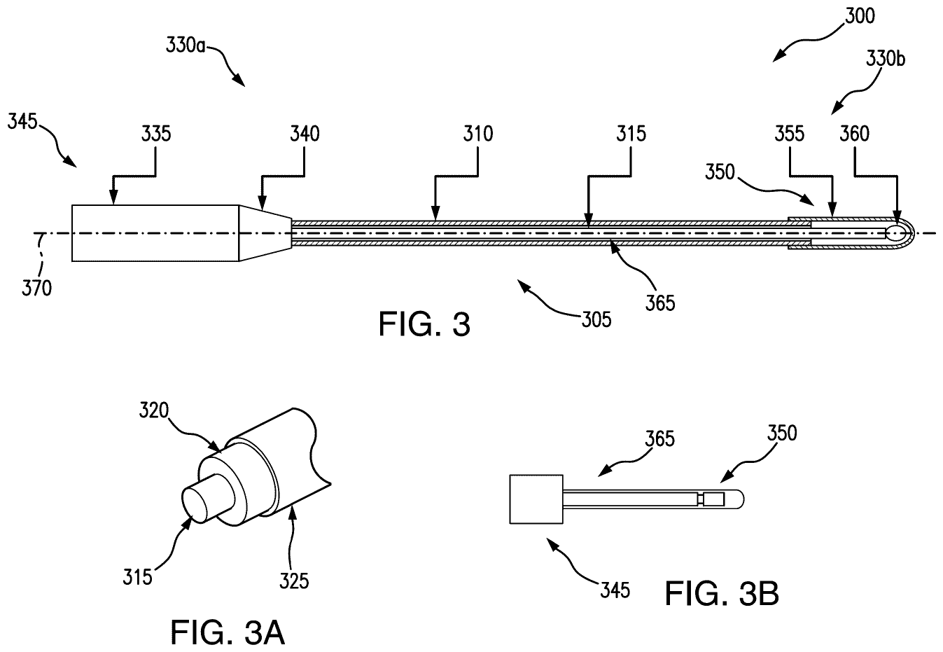

Referring now to FIGS. 2A-2B, a nasocatheter 205, e.g., a nasojejunal catheter, may be inserted into a patient via a nasal cavity 210 into a patient's stomach 105. For example, when the nasocatheter 205 is in the patient's stomach 105 near the pylorus 110, the nasocatheter 205 may further advance into the patient's gastrointestinal system by peristalsis, the involuntary constriction and relaxation of muscles of the intestine 215a, 215b, . . . 215n, to create wavelike movements that advance contents of the intestine forward as indicated by arrows 220. The nasocatheter 205 may have indicators, such as reference markings, for visualizing a length of the nasocatheter 205 inserted into a patient. In some embodiments, fluoroscopy, an electromagnetic tracking system, or some combinations thereof, or the like, may be used for visualization. In some embodiments, at least a portion of nasocatheter 205 may be radiopaque, so that a medical professional may visualize placement and to confirm location into the jejunum 120 by x-ray.

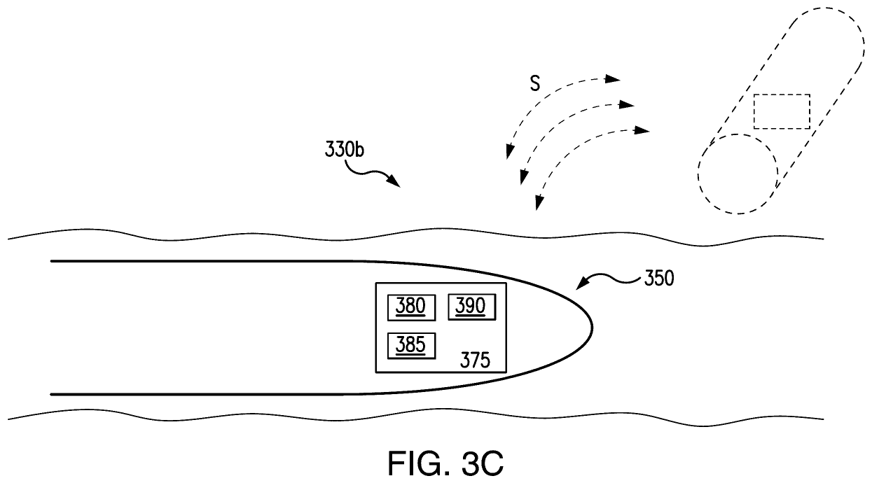

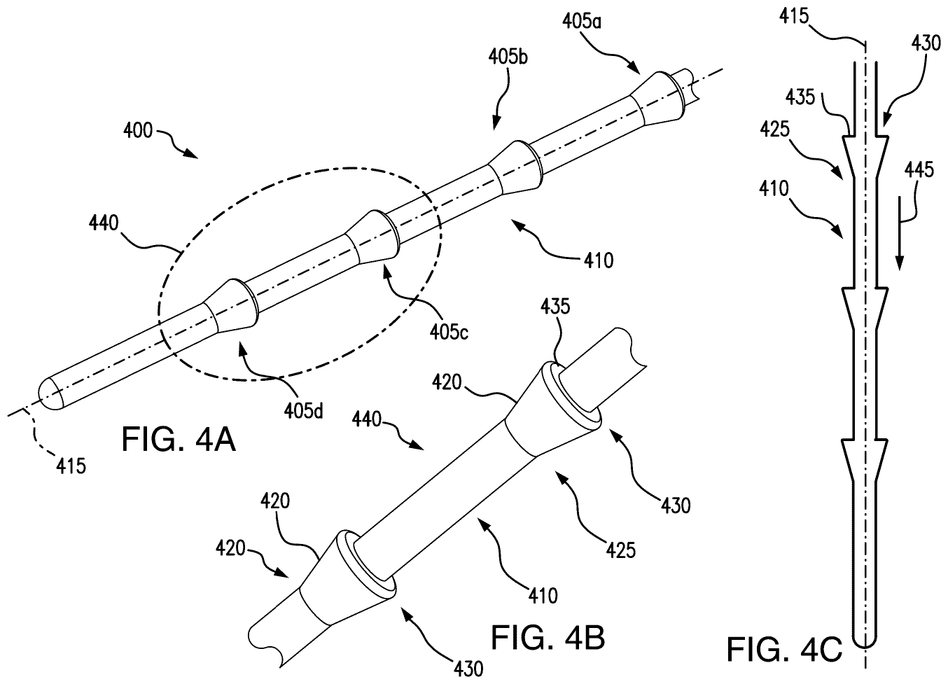

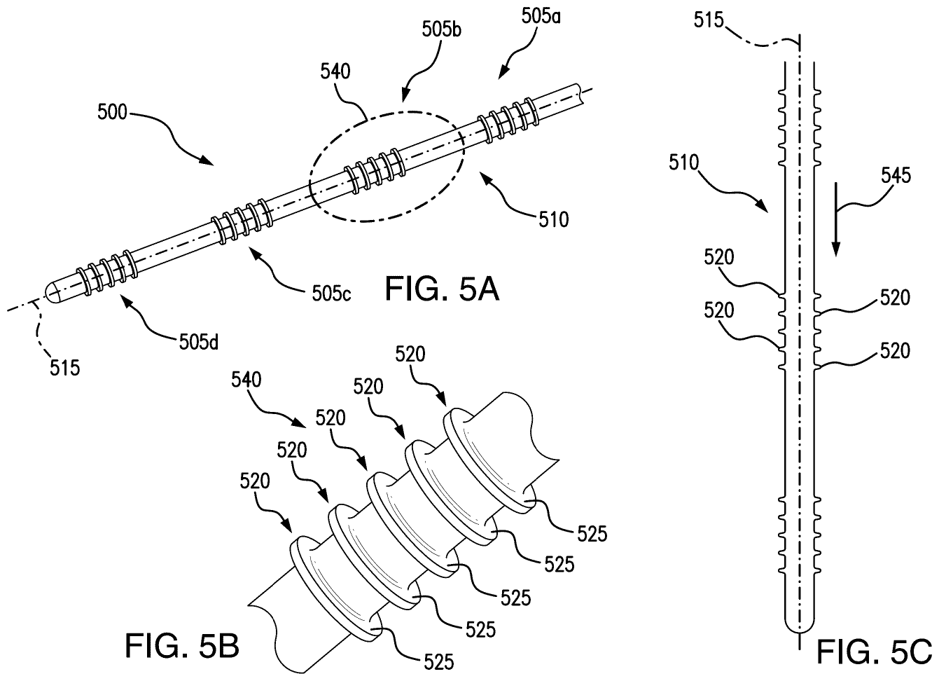

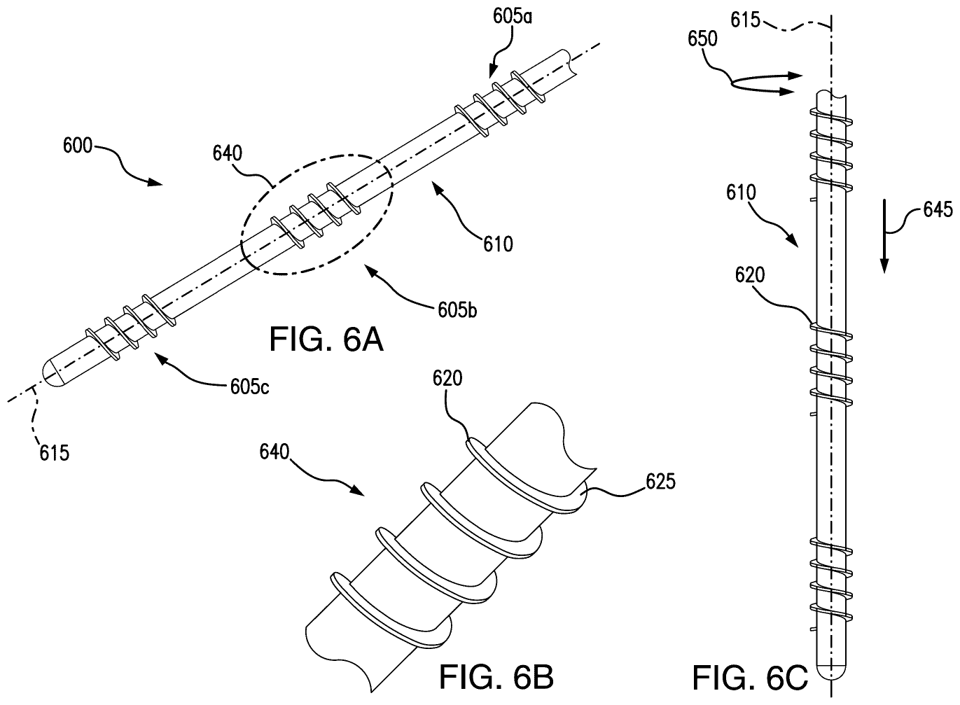

As shown in FIG. 3, an exemplary embodiment of a nasocatheter 300 in accordance with the present disclosure may allow for placement as described with respect to FIGS. 2A-2B. In some embodiments, the nasocatheter 300 may include a tube 305 extending along a longitudinal axis 370. The tube 305 may be flexible, for ease of insertion. In some embodiments, the nasocatheter 300 may be approximately 230 cm or more in length. The tube 305 may be overmolded and/or bonded to a fiber optic core 315. The nasocatheter 300 may include a locating element, e.g., so that a medical professional may locate a position of a distal end 330b of the nasocatheter 300 with an endoscope extending from the patient's gastric system. In embodiments, the locating element may be a light emitting element (see FIG. 3B) and/or a transmitter and/or receiver, configured for receiving a signal (see FIG. 3C).

In embodiments, e.g., illustrated in FIG. 3A, the overmolding 310 may include cladding 320 disposed around the fiber optic core 315 and covered by an outside jacket 325. A proximal end 330a of the nasocatheter 300 could be coupled to a light element, e.g., a light source 335, by a connector 340. In some embodiments, the connector 340 may be a universal connector, e.g., a SMA connector. The light source 335 may be a portable light source for direct connection to the fiber optic core 315 and/or the light source 335 may be an operating room (OR) light source.

In some embodiments and as shown in FIG. 3B, a proximal end 330a of the nasocatheter 300 may include a battery pack 345 for a locating element, e.g., a light emitting diode (LED). For example, a distal end 330b of the nasoscatheter may include a LED 350. The LED may be connectable to the battery pack 345 via lead-wires 365. The LED 350 may illuminate radially so that a medical professional may better visualize a location of the nasocatheter 300. The LED 350 may emit a constant source of light and/or may emit a blinking light. It may be understood that the light source 335 and/or the LED 350 may emit light at an intensity of up to approximately 35000 lux.

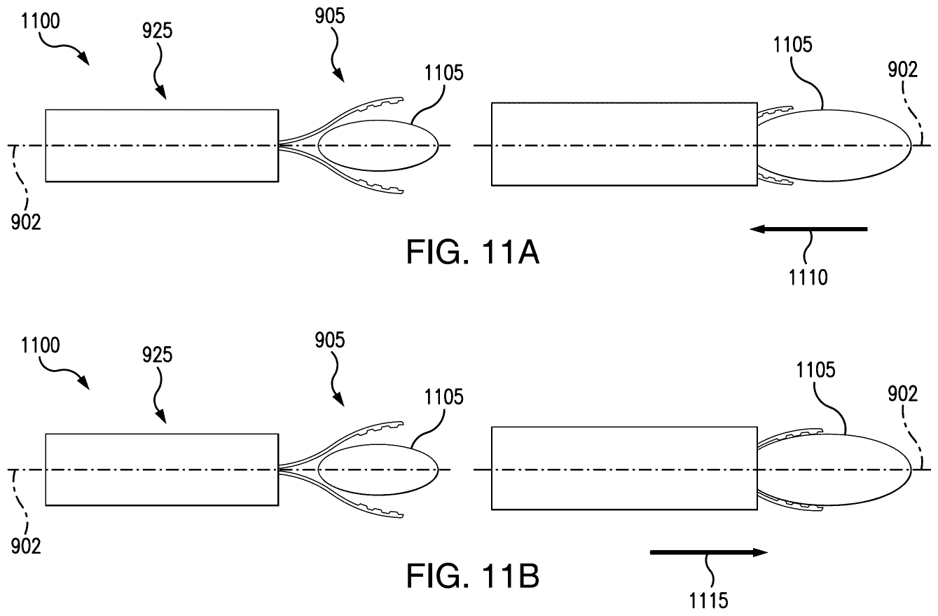

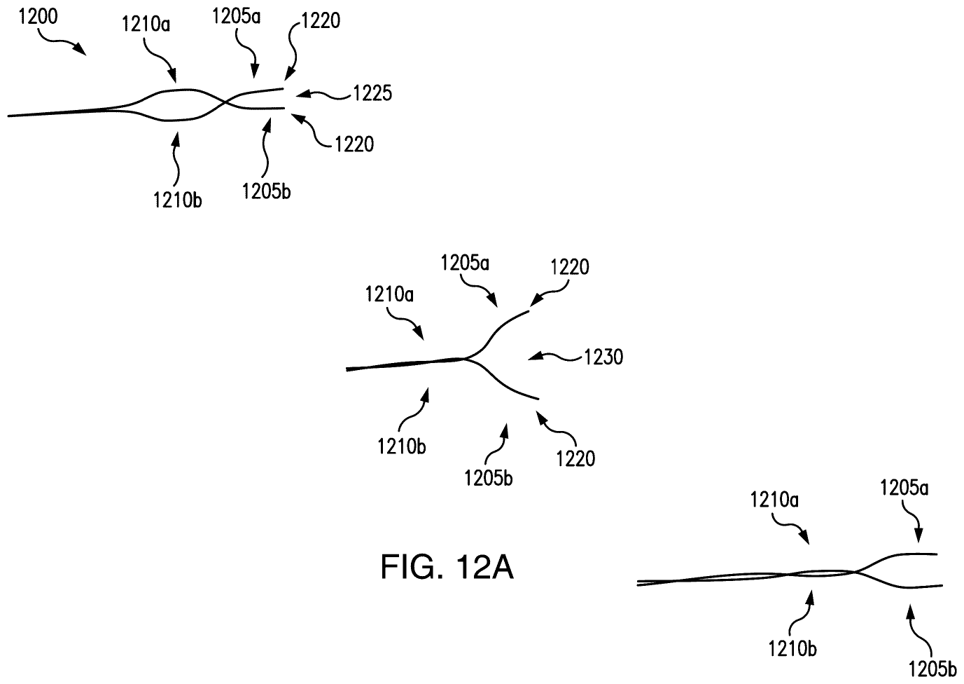

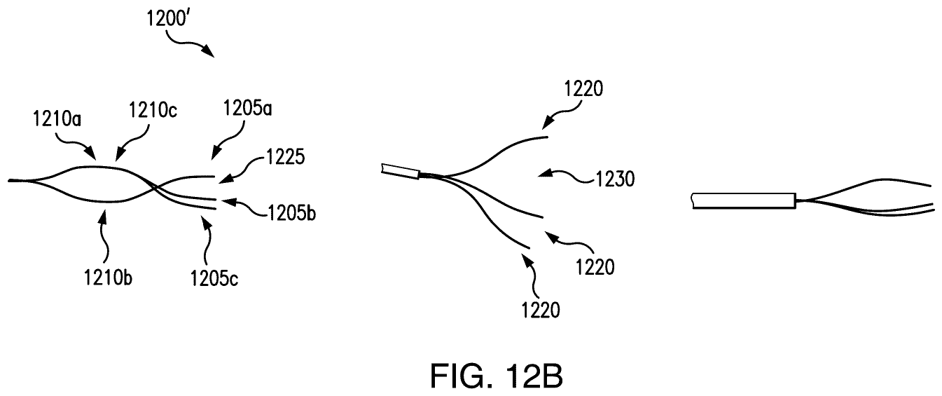

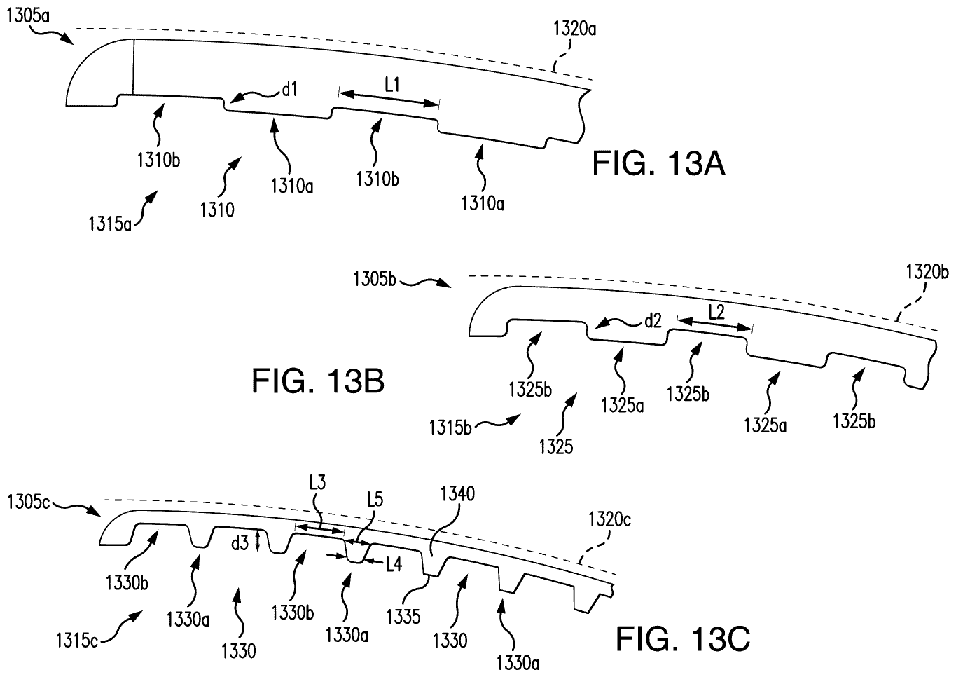

As further shown in FIG. 3, material 360 may be disposed at a distal end of a cover 355, extending over the locating element (e.g., fiber optic core 315 and/or the LED 350). In some embodiments, the cover 355 may be rounded and/or include an atraumatic tip, to minimize tissue damage as the nasocatheter 300 advances through a patient's anatomy (e.g., gastrointestinal system). In some embodiments, the material 360 may be a magnet, which may be magnetically attachable to graspers or other end effectors described below (see FIGS. 9A-13E), and in other embodiments, the material 360 may be a non-magnetic weight, to aide in advancement of the nasocatheter 300 by peristalsis. When an LED 350 is disposed at the distal end 330b of the nasocatheter 300, the material 360 may be disposed distally of the LED 350 so as to avoid interfering with light being emitted radially.

The locating element as a light source 335 and/or the LED 350 may allow a medical professional to visualize the distal end 330b of the nasocatheter 300 from outside the wall of the jejunum 120 by an endoscopic and/or laparoscopic visualization. As described below with respect to FIGS. 9A-13E, end effectors, e.g., graspers, may grasp an area illuminated by the nasocatheter 300 for creating an anastomosis in the desired position, e.g., at a selected distance from the pylorus 110. For example, an insertion may be made in the gastric wall, e.g., stomach, so that an endoscope or an endoscopic accessory may extend towards the jejunum for visualization of a light emitting from the nasocatheter 300.

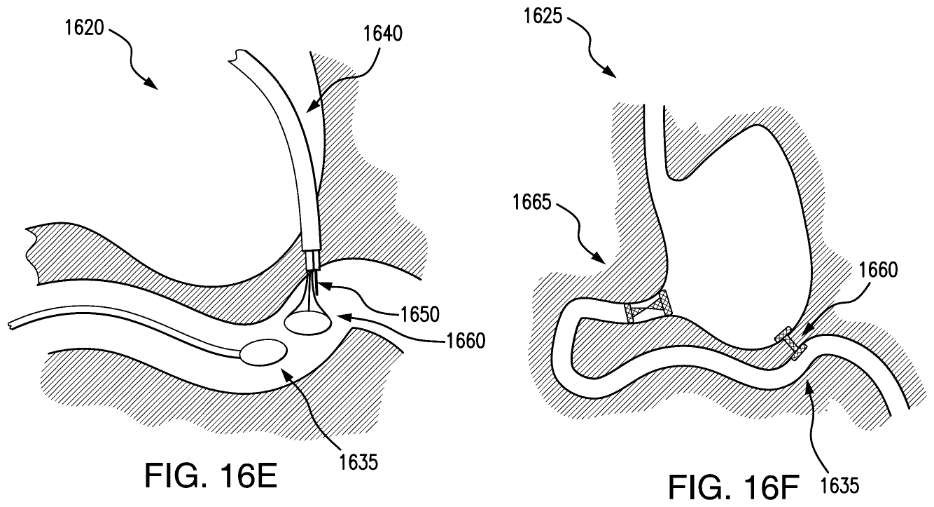

In another embodiment, as shown in FIG. 3C, a locating element may be a sensor 375, e.g., a micro-sensor, which may be disposed at the distal end 330b of the nasocatheter 300. The sensor 375 may be configured to emit a signal S, e.g., by a transmitter 380, that can be captured by a corresponding receiver attached to an endoscope and/or an accessory of an endoscope, such as an end effector and/or grasper, that is delivered to the patient's gastric system (e.g., stomach) (see FIG. 16C). In another embodiment, a receiver 385 may be disposed on the nasocatheter 300, and a corresponding transmitter may be attached on the endoscope and/or accessory of the endoscope. In some embodiments, the sensor 375 may be connected to a remote power source, e.g., disposed at a proximal end 330a of the nasocatheter 300, and in other embodiments, the sensor may include an internal power source 390.

A sensor configuration as described may be advantageous in that a medical professional may be able to manipulate the end effector and/or grasper to get closer to the targeted tissue, or loop of interest, before light emitted from the outside wall of jejunum is visible. The sensor 375 may be any type of sensor and/or micro-sensor, including but not limited to ultrasonic sensors, laser sensors, infrared (IR) sensors, electromagnetic sensors, hall-effect sensors, and/or magnetic reed switches. A weight of the sensor 375 on the nasocatheter may also aide in the advancement of nasocatheter 300 by peristalsis, e.g., supplementing and/or replacing the material 360.

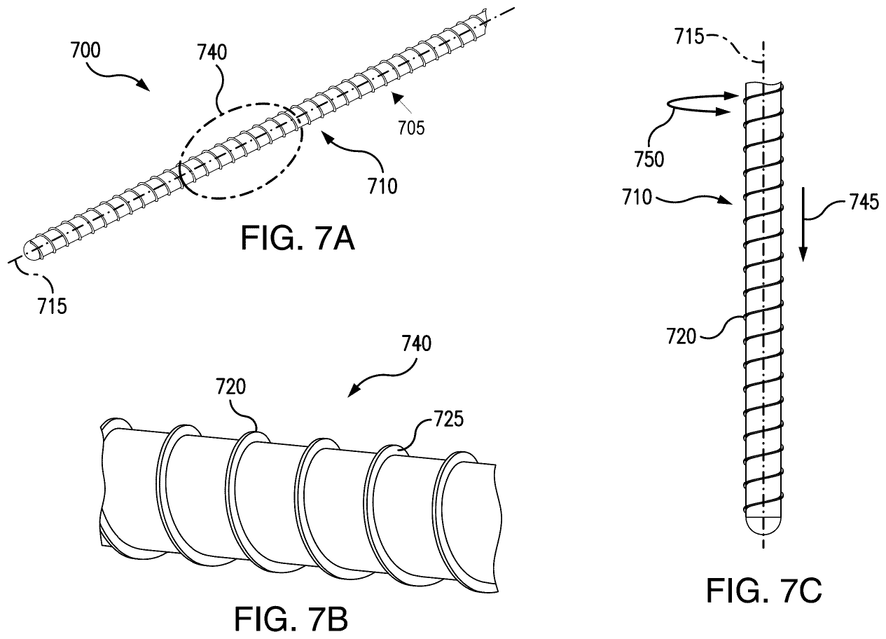

Referring now to FIGS. 4A-8C, exemplary embodiments of a nasocatheter in accordance with the present disclosure are shown. As described above, peristalsis may be utilized to advance a nasocatheter 205, 300 through a patient's gastrointestinal system to a desired position in the jejunum 120. A nasocatheter may include geometry on an outer surface to take advantage of the wave-like movements of peristalsis that may more quickly advance the nasocatheter in the intestine to the desired position. It is understood that the nasocatheter 400, 500, 600, 700, 800 may include identical features as the nasocatheter 300 except as described below.

As shown in FIGS. 4A-4C, a nasocatheter 400 may have one or more frustoconical sections 405a, 405b, . . . 405n disposed along an outer surface 410 and extending along a longitudinal axis 415. The frustoconical sections 405a, 405b, . . . 405n may be disposed along an entire length of the nasocatheter 400, or may be disposed in a clustered portion on the nasocatheter 400. Placement of the frustoconical sections 405a, 405b, . . . 405n may be determined by peristaltic movement of a patient's gastrointestinal system.

Each section may include a frustoconical portion 420, in which the frustoconical 420 includes a smaller diameter 425 distal of a larger diameter 430. The frustoconical portion 420 may be integrally formed with the nasocatheter 400, although in some embodiments, the frustoconical portion 420 may be attached to the nasocatheter, e.g., by a mechanical fastener, adhesive, and/or geometrical configuration such as a press fit, tongue and groove, and/or slot configuration. The smaller diameter 425 may be approximately the diameter of the nasocatheter 400 and flush with the surface 410 of the nasocatheter 400, and the larger diameter 430 may extend radially from the surface 410 of the nasocatheter 400. The frustoconical portion 420 may include a planar surface 435 extending substantially perpendicular to the longitudinal axis 415 connecting the larger diameter 430 to the surface 410 of the nasocatheter 400 (see reference numeral 440 and FIG. 4B). As the intestinal walls contract and expand during peristalsis creating the wave-like movement (see FIG. 2B), the walls may contact the planar surface 435 to provide a force in a forward, distal direction as indicated by arrow 445 and substantially parallel to the nasocatheter 400, thereby maximizing the force to advance the nasocatheter 400. Additionally, the wave-like movement may include forward moving and/or propagation for advancing the nasocatheter 400. The frustoconical portion 420 of the nasocatheter 400 may minimize and/or prevent any backward motion as a result of the peristaltic motion (e.g., retro-peristalsis or reverse peristalsis in instances of vomiting and/or other infections).

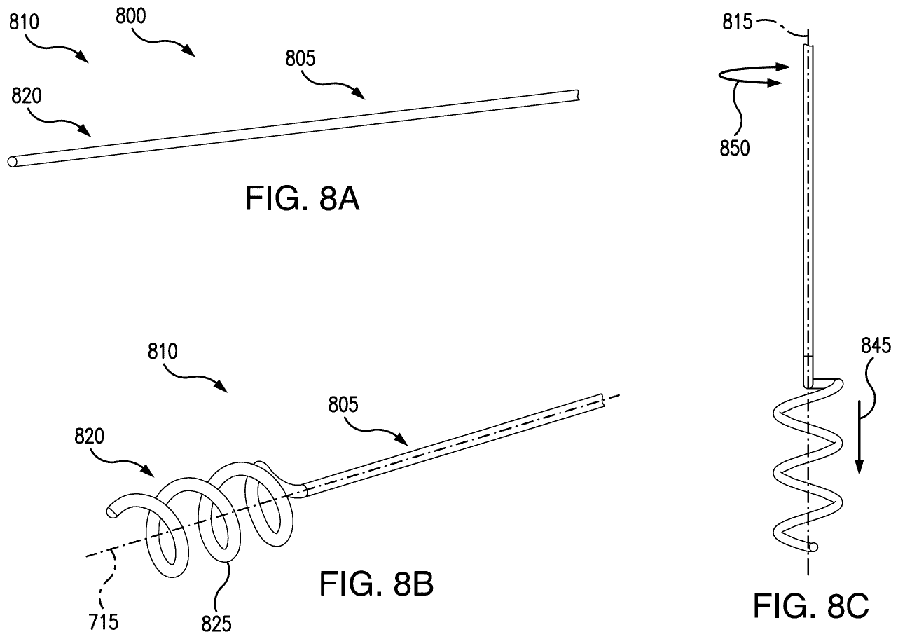

Referring now to FIGS. 5A-5C, a nasocatheter 500 may have one or more protrusions, or sets of protrusions 505a, 505b, . . . 505n extending radially outward from a surface 510 of the nasocatheter 500 and disposed along a longitudinal axis 515. The sets of protrusions 505a, 505b, . . . 505n may be disposed along an entire length of the nasocatheter 500, or may be disposed in a clustered portion on the nasocatheter 500. Placement of the sets of protrusions 505a, 505b, . . . 505n may be determined by peristaltic movement of a patient's gastrointestinal system.

The sets of protrusions 505a, 505b, . . . 505n may include a plurality of protrusions 520, which may each be a circular section and/or ridge. Each protrusion 520 may extend radially from the surface 510 and substantially perpendicular from the longitudinal axis 515, although in other embodiments the protrusions 520 may extend at any angle relative to the surface 510 of the nasocatheter 500. In some embodiments, the protrusions 520 may be spaced equidistantly from each other along the longitudinal axis 515, in each set of protrusions 505a, 505b, . . . 505n, although in other embodiments, the projections may be variably spaced apart from each other. The protrusions 520 may be integrally formed with the nasocatheter 500, although in some embodiments, the protrusions 520 may be attached to the nasocatheter 500, e.g., by a mechanical fastener, adhesive, and/or geometrical configuration such as a press fit, tongue and groove, and/or slot configuration. Each protrusion 520 may have a proximal surface side 525, so that the intestinal wall may contact the surface sides 525 during contractions (see reference numeral 540 and FIG. 5B). For example, as mentioned above with the frustoconical configuration, during peristalsis the intestinal walls contract and contact the surface sides 525 to provide a force in a forward direction as indicated by arrow 545 and substantially parallel to the nasocatheter 500, thereby maximizing the force to advance the nasocatheter 500.

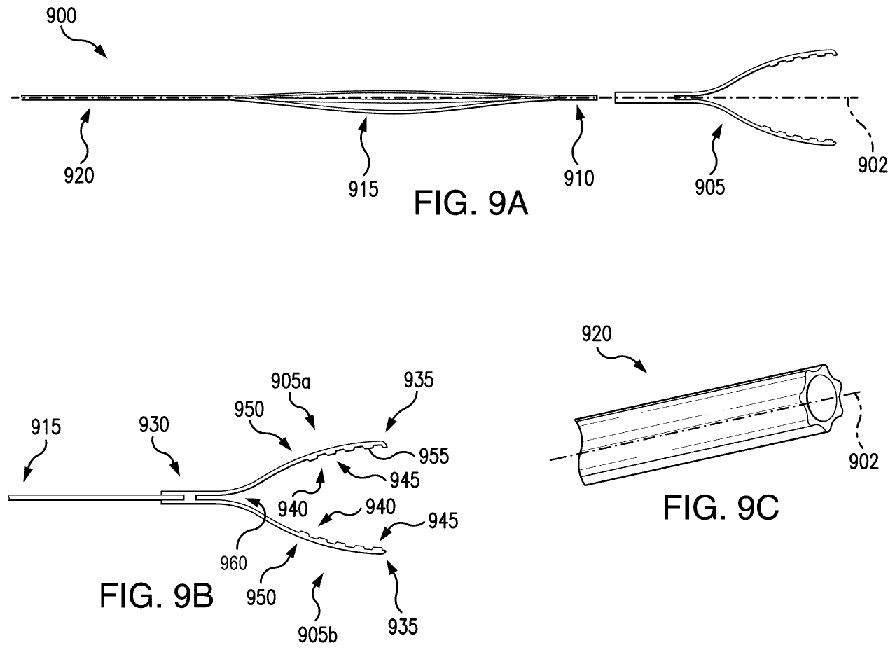

Referring now to FIGS. 6A-6C, a nasocatheter 600 may have one or more helical features 605a, 605b, . . . 605n, extending radially outward from a surface 610 of the nasocatheter 600 and disposed along a longitudinal axis 615. The helical features 605a, 605b, . . . 605n may be disposed along an entire length of the nasocatheter 600, or may be disposed in a clustered portion on the nasocatheter 600. Placement of the helical features 605a, 605b, . . . 605n may be determined by peristaltic movement of a patient's gastrointestinal system.

The helical features 605a, 605b, . . . 605n may include a helical protrusion 620, extending radially from the surface 610, e.g., a screw thread. In embodiments having a helical feature such as a screw thread, a higher pitch of the helical features may correlate to more forward movement (e.g., in direction of arrow 645) as long as peristalsis allows for continuous engagement of the screw thread. It may be understood that if a pitch is too high there may not be continuous engagement with the screw thread (e.g., the intestinal walls may have intermittent engagement and disengagement with the screw thread, which may result in less forward movement or less efficient forward movement.

The helical protrusion 620 may be integrally formed with the nasocatheter 600, although in some embodiments, the helical protrusion 620 may be attached to the nasocatheter 600, e.g., by a mechanical fastener, adhesive, and/or geometrical configuration such as a press fit, tongue and groove, and/or slot configuration. The helical protrusion 620 may have a proximal surface side 625, so that the intestinal wall may contact the surface side 625 during contractions (see reference numeral 640 and FIG. 6B). For example, during peristalsis the intestinal walls contract and may contact the surface side 625 to provide a force in a forward direction as indicated by arrow 645 and substantially parallel to the nasocatheter 600, thereby maximizing the force to advance the nasocatheter 600. As the intestinal walls contract and advance the nasocatheter 600 in a direction indicated by arrow 645, the helical protrusion 620 may also result in the nasocatheter 600 rotating about the longitudinal axis 615 as shown by arrow 650, e.g., as a corkscrew.

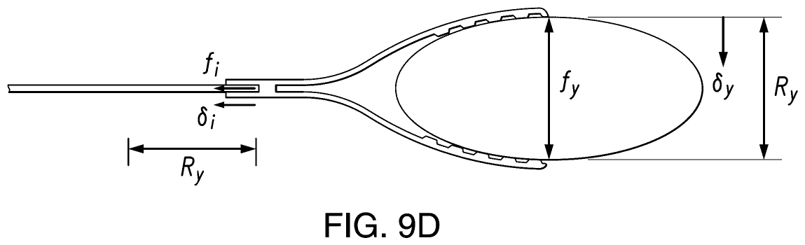

Referring now to FIGS. 7A-7C, a nasocatheter 700 may have a continuous helix 705, e.g., a screw thread, extending radially outward from a surface 710 of the nasocatheter 700 and disposed along a longitudinal axis 715. The helix 705 may be disposed along an entire length of the nasocatheter 700. The helix 705 may be integrally formed with the nasocatheter 700, although in some embodiments, the helix 705 may be attached to the nasocatheter 700, e.g., by a mechanical fastener, adhesive, and/or geometrical configuration such as a press fit, tongue and groove, and/or slot configuration.

The helix 720 may have a proximal surface side 725, so that the intestinal wall may contact the surface side 725 during contractions (see reference numeral 740 and FIG. 7B). For example, during peristalsis the intestinal walls contract and may contact the surface side 725 to provide a force in a forward direction as indicated by arrow 745 and substantially parallel to the nasocatheter 700, thereby maximizing the force to advance the nasocatheter 700. As the intestinal walls contract and advance the nasocatheter 700 in a direction indicated by arrow 745, the helix 720 may also result in the nasocatheter 700 rotating about the longitudinal axis 715 as shown by arrow 750, e.g., as a corkscrew.

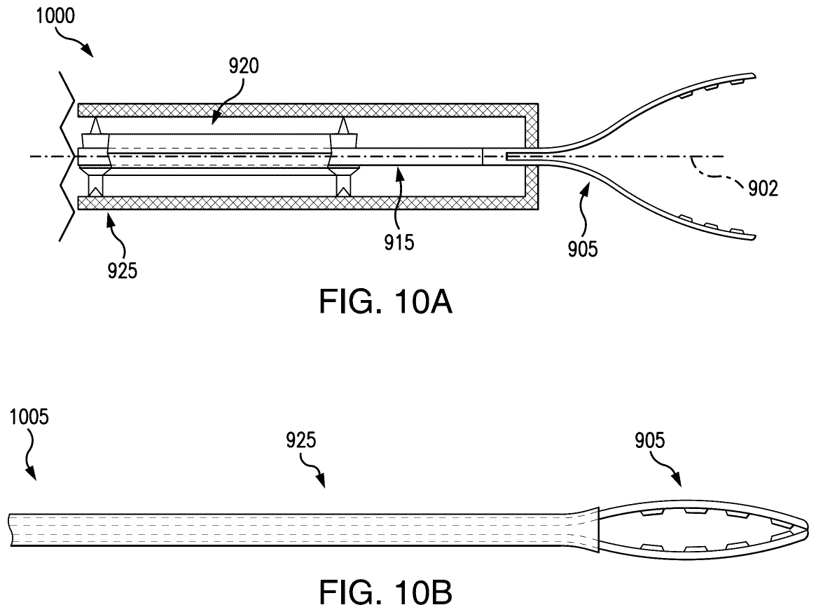

Referring now to FIGS. 8A-8C, a nasocatheter 800 may include a tube 805 extending along a longitudinal axis 815. The tube 805 may be injection molded and/or extruded over a self-expanding or shape memory wire (e.g., nitinol) or mandrel and then heat set to a spiral shape over at least a portion of the tube 805. For example, a distal end 810 may include a spiralable portion 820. During insertion of the nasocatheter 800 into a patient's stomach, the tube 805 may be substantially straight, e.g., by a guidewire (not shown) internal to the tube 805 (see FIG. 8A). When the medical professional determines that the nasocatheter 800 is in a desired location, e.g., in the pylorus 110, the guidewire may be removed from the tube 805, thereby allowing the spiralable portion 820 at the distal end 810 to expand into a spiral 825 (see FIGS. 8B-8C).

For example, during peristalsis the intestinal walls contract and may contact the spiral 825 to provide a force in a forward direction as indicated by arrow 845 and substantially parallel to the nasocatheter 800, thereby maximizing the force to advance the nasocatheter 800. As the intestinal walls contract and advance the nasocatheter 800 in a direction indicated by arrow 845, the spiral 825 may also result in the nasocatheter 800 rotating about the longitudinal axis 815 as shown by arrow 850, e.g., as a corkscrew.

When the nasocatheter 205, 300, 400, 500, 600, 700, 800, is in the desired position, e.g., at a selected distance from the pylorus 110, an endoscope may be inserted into a patient's stomach 105 for deploying additional accessories. For example, an endoscope may include a channel for deploying an end effector for locating and grasping a proximal area of the jejunum (see FIGS. 9A-11B).

Referring now to FIGS. 9A-9D, an exemplary embodiment of a grasping system 900 in accordance with the present disclosure is shown. An exemplary embodiment of a grasping system may be able to grasp a desired portion of a small bowel without perforating tissue, e.g., an end effector may be atraumatic to minimize tissue damage. The end effector may be flexible for advancement through an endoscope, and have a length sufficient for extending to the desired location in the jejunum.

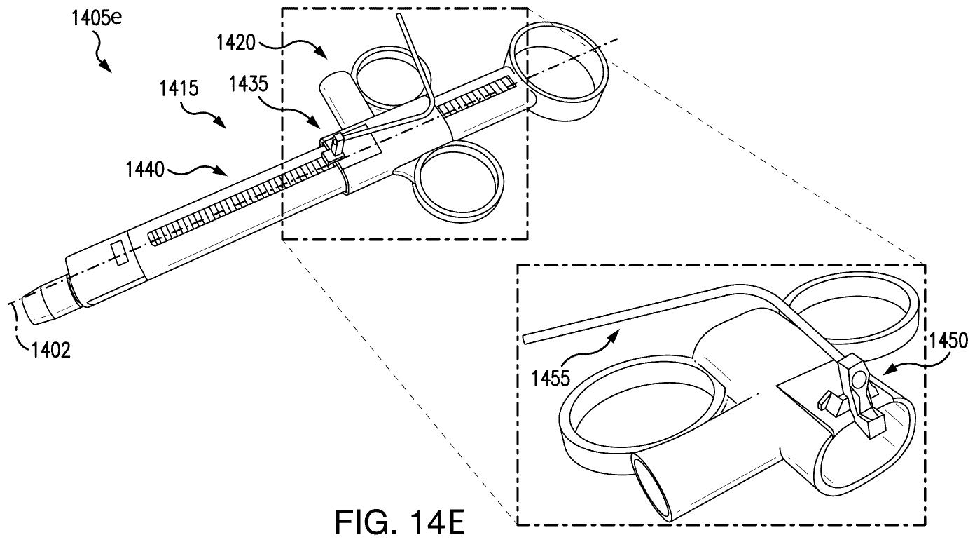

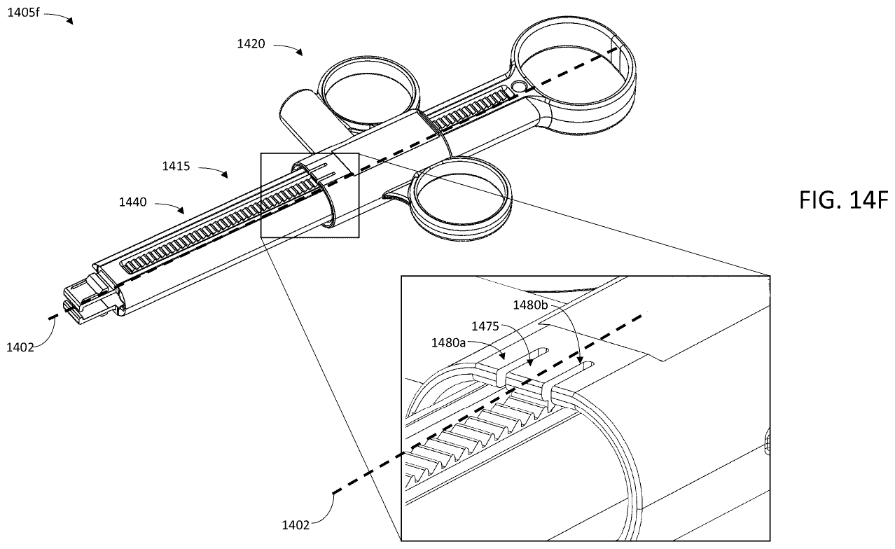

An end effector 905 may include arms 905a, 905b, . . . 905n disposed at a distal end of the end effector 905 and extending longitudinally along a longitudinal axis 902. In some embodiments, the end effector 905 may include two arms 905a, 905b, and in other embodiments three or more arms 905a, 905b, . . . 905n (see also FIGS. 12A-12B). The arms 905a, 905b may extend from a common proximal joint 930 of the end effector 905, and may be connectable to a drive wire 915 at joint 910. For example, the common proximal joint 930 may be a yoke, e.g., a single connection of the first and second arms 905a, 905b, from a unitary proximal end. Arms extending from a unitary proximal end may be advantageous in maintaining a constant downward force on respective distal ends of each arm 905a, 905b while maintaining an opening between the arms. Additionally, distal ends of the arms 905a, 905b may begin closing when the unitary proximal end is compressed, which may be advantageous for uniform closing and force distribution on grasped tissue from both arms 905a, 905b. In some embodiments, the arms 905a, 905b may be integrally formed from the unitary proximal end, which may ensure alignment of the arms relative to each other. Mating of respective protrusions (e.g., teeth) from each arm 905a, 905b may also be ensured by alignment of the arms 905a, 905b, which may be advantageous for holding very thin tissue portions without causing tissue damage (e.g., perforations).

In some embodiments, the drive wire 915 may be a single wire, double wire, or three wires, e.g., a triple-wire drive system, and the joint 910 may be a crimp connection for joining the wires and connecting to the common proximal joint 930 of the end effector 905. In some embodiments, the common proximal joint 930 may be joined to the drive wire 915 by laser welding, adhesive, and/or mechanical fastener. For example, the joint 910 may be joined to the end effector 905 through a hole (not shown) of the common proximal joint 930 of the arms 905a, 905b.

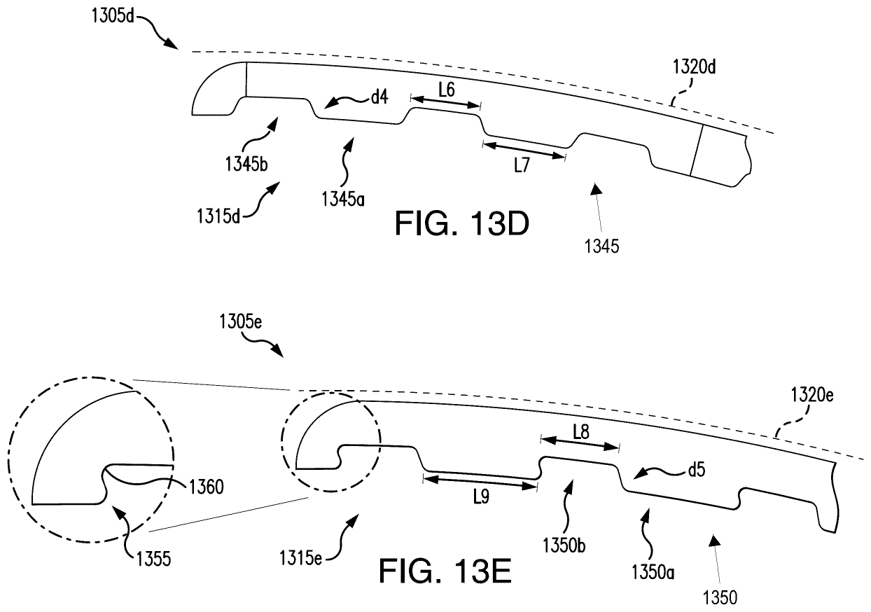

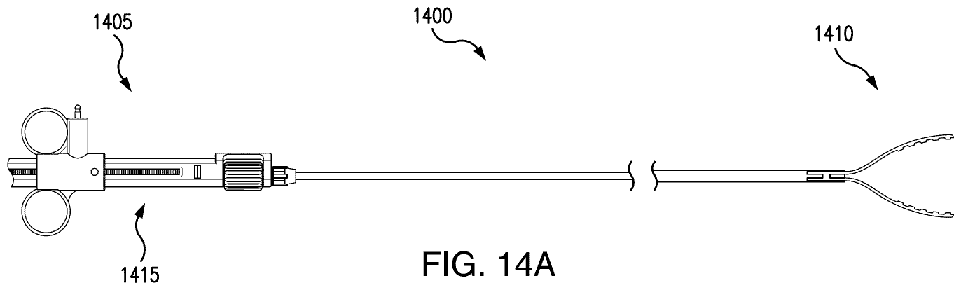

The end effector 905 and drive wire 915 may be disposed within a sheath 925 extending along and disposed coaxial to the longitudinal axis 902 (see FIGS. 10A-10B), and in some embodiments, tubing 920 may provide internal support to the sheath 925. The sheath 925 may be formed of a flexible material including by not limited to braided Pebax, PTFE, Teflon, and the like, and in some embodiments, may be reinforced with metal braiding, e.g., stainless steel. The tubing 920 may be discrete tubular portions, e.g., floating tubes, to form a rigid section of the flexible sheath 925 and extending along and coaxial to the longitudinal axis 902. For example, each tubing 920 portion may be disposed along the sheath 925 and may be movable freely along the longitudinal axis 902. Tubing 920 may take up any gaps between the drive wire 915 and the sheath 925, e.g., to stabilize the wire for rotation without buckling and/or for efficiently transmitting torque and/or actuation force along the length of the wire. In some embodiments, the tubing 920 may be star tubing, having a plurality of sides to prevent rotation and to allow for compression. For example, star tubing 920 may be hexagonal and/or heptagonal. In some embodiments, the tubing 920 may be formed of a polytetrafluoroethylene (PTFE) material.

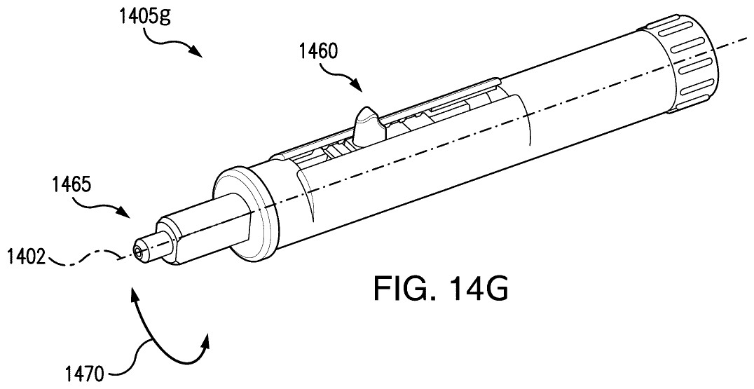

The arms 905a, 905b may have an outer surface 950, e.g., a surface on an outer circumferential portion of the arc shape 940, and an inner surface 945, e.g., a surface on an inner circumferential portion of the arc shape 940. The inner surface 945 may include one or more projections 955, e.g., teeth. It is understood that all arms 905a, 905b, . . . 905n on an end effector 905, may include projections 955, although in other embodiments one arm (e.g., either 905a or 905b) may include projections 955 on the inner surface 945 while the other of the arm 905a or 905b does not include any projections 955. Projections on the inner surface 945 of the arms 905a, 905b may increase surface area for gripping tissue to spread out forces applied to tissue, thereby minimizing a risk of perforating tissue during grasping. The projections 955 may be any shape and size for distributing gripping forces in an atraumatic manner (see FIGS. 13A-13E).

In embodiments, the arms 905a, 905b may be hingeless, e.g., the end effector 905 may be formed of a self-expanding or shape memory material such as nitinol. For example, the end effector may have arms that are manipulatable without including a mechanical hinge fastener for movement relative to each other such as scissors. In systems having a scissor-like hinge, when the arms are manipulated to a closed position, forces applied to tissue at the distal end are dependent on the angle between the arms 905a, 905b at the hinge, so that a greater force maybe applied at the distal end of the arms as opposed to the proximal end at the handle. Additionally, as the hinge manipulates the arms to a closed position, tissue may be pushed out from between the arms, resulting in a smaller volume of tissue gripped by the end effector. A smaller volume of tissue gripped between the arms of the end effector, combined with potential higher forces being exerted by a medical professional with reduced tactile feedback may increase a risk of perforation or other tissue damage. In accordance with the present disclosure, and as shown in FIG. 9D, arms 905a, 905b having a common proximal joint 930 may allow for a force applied at the proximal end of the handle by the medical professional to be dependent on a force gripping the tissue at the distal end of the end effector 905. This correlation may also improve tactile feedback for the medical professional, to minimize a risk of tissue damage (e.g., perforation). In some embodiments, the arms 905a, 905b may extend from the common proximal joint 930 substantially parallel to each other and spaced apart, as indicated at reference numeral 960. During closure of the arms 905a, 905b, a gap may allow for tissue to conform to the gap without becoming pinched or otherwise damaged.

A self-expanding or shape memory material such as nitinol may have higher elasticity and may therefore allow for a greater opening span as opposed to other known graspers. Additionally, a self-expanding or shape memory material such as nitinol may allow for the arms 905a, 905b to more easily be constrained within a catheter.

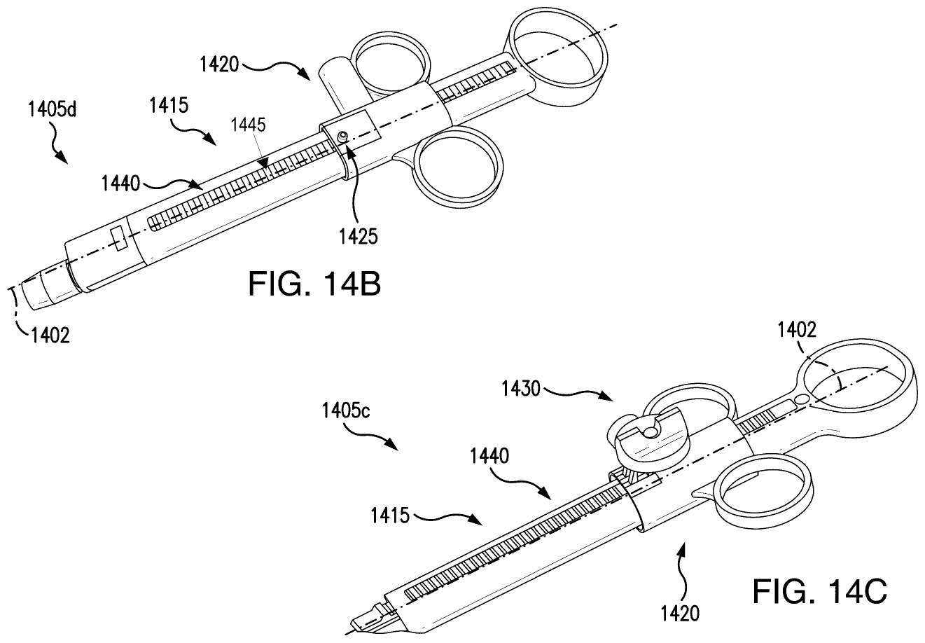

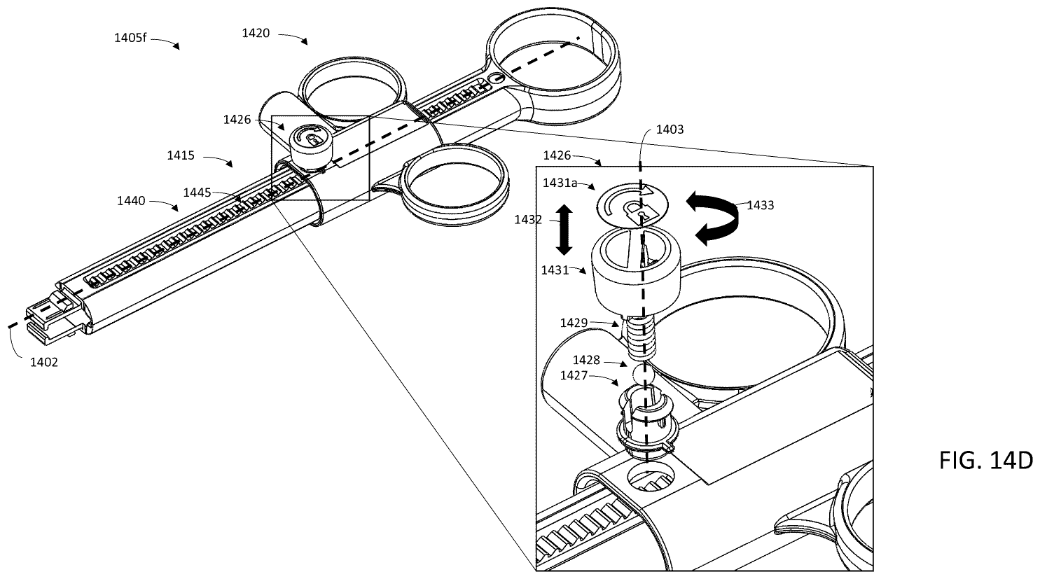

In a constrained position 1005, the arms 905a, 905b may be closed (e.g., FIG. 10B). In embodiments, the arms 905a, 905b may be fully constrained within the sheath 925, such that corresponding protrusions on each arm 905a, 905b may be mated. In an unconstrained position 1000 as shown in FIG. 10A, the arms 905a, 905b may be substantially open. For example, the nitinol may be heat set so that the arms 905a, 905b have a spring force to expand the arms 905a, 905b in an open position when unconstrained. The arms 905a, 905b may be formed in an arc shape 940 (see FIG. 9B) and disposed relative to each other so that in the constrained position only distal tips 935 of each arm 905a, 905b contact each other. Additionally, when the arms 905a, 905b are manipulated into a constrained position, the arms 905a, 905b may collapse, or begin to close, at the distal tip 935 first, thereby encompassing tissue within the arc shape 940. For example, an oval shape may be formed between the first and second arms 905a, 905b by the arc shape 940. This arc shape 940 may be advantageous so that tissue may be grasped and pulled in a proximal direction by a medical professional (see FIGS. 11A-11B). For example, tissue may be grasped and held within the arc shape 940 so that the medical professional may achieve a grip of the selected tissue while minimizing risk of tissue perforation. Additionally, the arc shape 940 when formed of a self-expanding or shape memory material such as nitinol may allow for a constant gripping force for various sized tissue. For example, material properties of nitinol are such that stress/strain characteristics allow for high gripping forces without damaging (e.g., perforating) tissue.

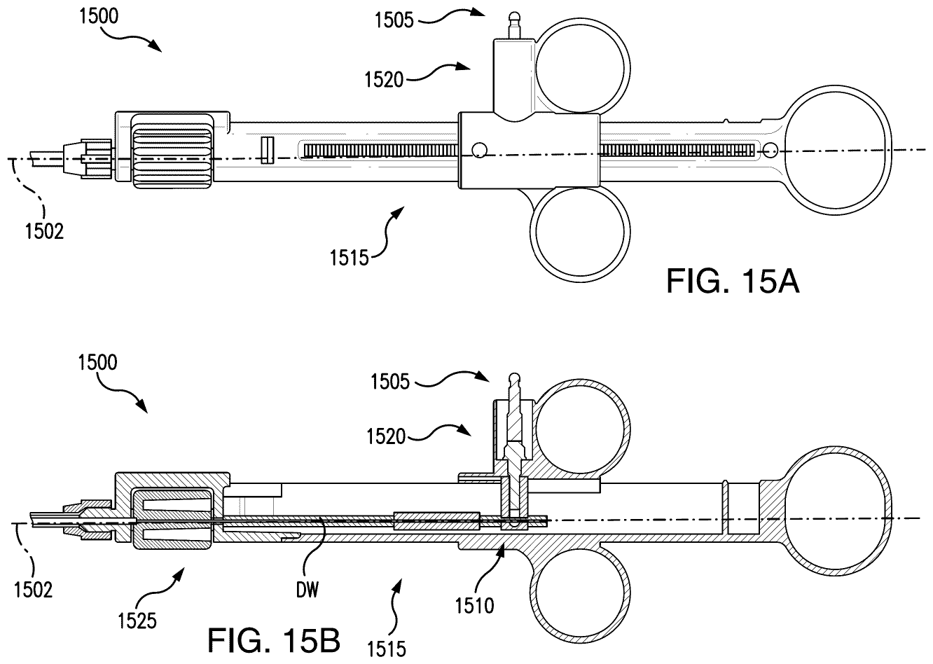

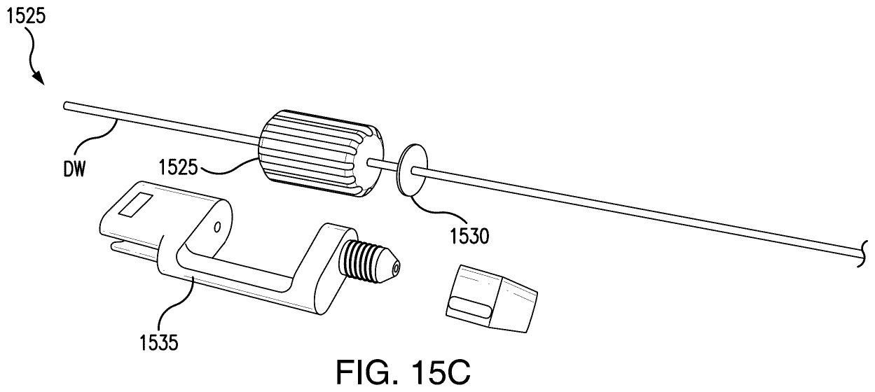

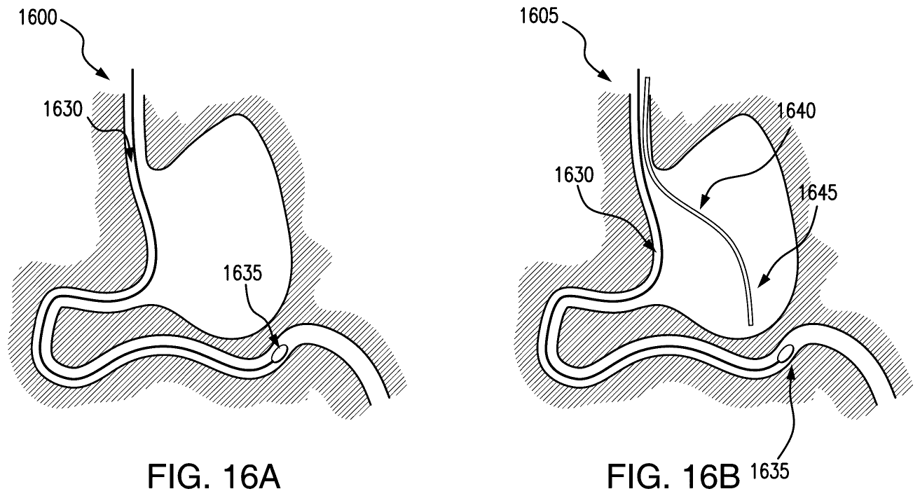

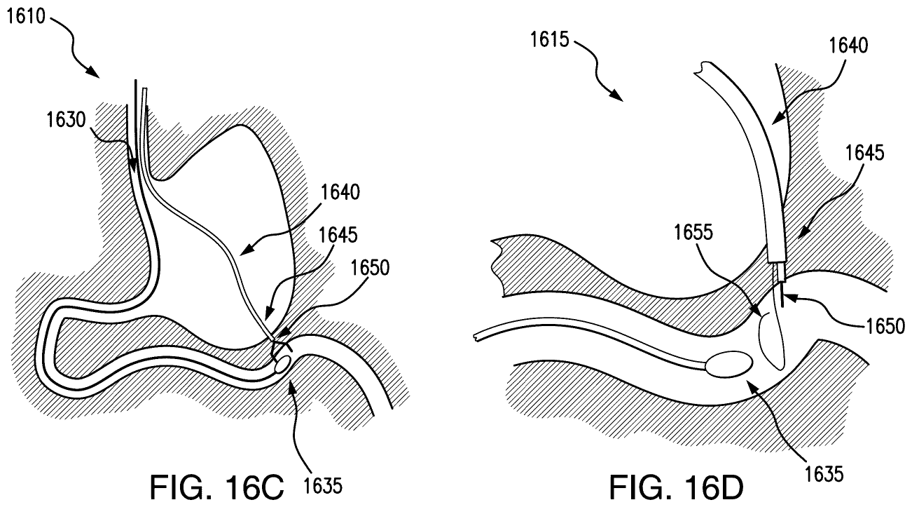

FIGS. 11A-11B illustrate exemplary embodiments of a closure of an end effector 905 according to the present disclosure, at a distal end 1100 of the grasping system 900. When the arms 905a, 905b are manipulated from an unconstrained position to a constrained position, the arc shape 940 of the arms 905a, 905b may close at the distal tip 935 first. In embodiments, the medical professional may pull the drive wire 915 in a proximal direction along the longitudinal axis 902 indicated at arrow 1110, to manipulate the arms 905a, 905b, thereby drawing the arms 905a, 905b into the sheath 925, as shown in FIG. 11A. For example, as will be described below with respect to FIGS. 14A-15C, the sheath 925 may be fixed to a handle disposed at a proximal end of the grasping system 900, for handling by a medical professional outside of the patient's body. The drive wire 915 may be connected to a dynamic portion of the handle for actuation of the end effector 905 with respect to the sheath 925. This configuration may be advantageous to provide tactile feedback to the medical profession, to indicate when the end effector 905 has a sufficient grip on tissue 1105.

In some embodiments, the medical professional may close the arms 905b, 905b by extending the sheath 925 in a distal direction along the longitudinal axis 902 as indicated at arrow 1115, leaving the end effector 905 in the desired position as the sheath closes the arms 905a, 905b around tissue, as shown in FIG. 11B. For example, as will be described below with respect to FIGS. 14A-15C, the drive wire 915 may be fixed to a handle and the sheath 925 may be connected to a dynamic portion of the handle for actuation of the end effector 905 with respect to the drive wire 915. This configuration may be advantageous to minimize a risk of slippage of tissue 1105 from the end effector 905 during actuation. It is understood that in this configuration the sheath 925 may absorb higher forces as it is advanced distally over the end effector 905, and thus may need additional reinforcements, e.g., increased column strength, to prevent collapse.

Although some embodiments illustrate arms 905a, 905b having an arc shape 940, other arms shapes are envisioned. Referring now to FIGS. 12A-12B, an end effector 1200 may have two arms 1205a, 1205b in a wave-like shape. For example, a self-expanding or shape memory material such as nitinol may heat set arms 1205a, 1205b in a wave pattern opposite of each other. As shown in FIG. 12A, a peak 1210a of arm 1205b may be aligned to a valley 1210b of arm 1205a, thereby positioning distal ends 1220 of each arm 1205a, 1205b at a predefined opening 1225. When actuated (see FIGS. 11A-11B), the peak 1210a and valley 1210b may be drawn together so that the distal ends 1220 may expand to a wider opening 1230 than opening 1225. As the end effector 1200 is actuated further, e.g., a sheath advances over the arms 1205a, 1205b and/or the arms 1205a, 1205b retract within the sheath, the opening may be closed to grasp tissue between the arms 1205a, 1205b. As shown in FIG. 12B, three arms 1205a, 1205b, 1205c may form a “tri-wave” configuration, in which a self-expanding or shape memory material such as nitinol may be heat set in a wave-like shape similar to the arms 1205a, 1205b, and may operate in a similar manner as in FIG. 12A. In some embodiments, a tri-wave configuration may be advantageous for a more balanced grip of desired tissue. For example, peak 1210a, valley 1210b, and peak 1210c may be drawn together during actuation to open distal ends 1220 to a wider opening 1230. As the end effector 1200′ is actuated further, e.g., a sheath advances over the arms 1205a, 1205b, 1205c and/or the arms 1205a, 1205b, 1205c retract within the sheath, the opening may be closed to grasp tissue between the arms 1205a, 1205b, 1205c. The end effector 1200, 1200′ may be advantageous for creating a wider opening of arms than other configurations for a typical channel opening (e.g., approximately 2-3 mm).

As described above, the inner surface 945 of the arm 905a, 905b may include one or more projections 955, which may allow for sufficient gripping force on the selected tissue by increasing surface area. Referring now to FIGS. 13A-13E, projections may be configured in various exemplary embodiments in accordance with the present disclosure. As shown in FIG. 13A, arm 1305a may include castellations 1310, e.g., a series of projections 1310a and grooves 1310b, on an inner surface 1315a of the arm 1305a. The projections 1310a and grooves 1310b may be similarly shaped and sized so that the castellations 1310 are consistent along the inner surface 1315a of the arm 1305a, although in other embodiments, the projections 1310a may be sized and/or shaped differently from the grooves 1310b, and/or individual projections 1310a and/or grooves 1310b may be shaped and/or sized differently. For example, projections and grooves 1310a, 1310b may provide atraumatic soft jaws for the arm 1305a. In embodiments, edges of the castellations may be filleted, chamfered, and/or rounded to further minimize tissue damage. In some embodiments, the edges of the projections and grooves 1310a, 1310b may be substantially perpendicular along a curvature 1320a of the arm 1305a. For example, the arm 1305a may have a curvature, or arc shape, similar to the arc shape 940 of arms 905a, 905b. It is also envisioned that edges of the projections and grooves 1310a, 1310b may extend at angles relative to the curvature 1320a of the arm 1305a.

As shown in FIG. 13B, arm 1305b may include castellations 1325, e.g., a series of projections 1325a and grooves 1325b, on an inner surface 1315b of the arm 1305b. The projections 1325a and grooves 1325b may be similarly shaped and sized so that the castellations 1325 are consistent along the inner surface 1315b of the arm 1305b, although in other embodiments, the projections 1325a may be sized and/or shaped differently from the grooves 1325b, and/or individual projections 1325a and/or grooves 1325b may be shaped and/or sized differently. For example, the projections and grooves 1325a, 1325b may be rectangular with rounded teeth, e.g., alligator jaws for arm 1305b. In some embodiments, edges of the castellations may be filleted, chamfered, and/or rounded to further minimize tissue damage. In some embodiments, the edges of the projections and grooves 1325a, 1325b may be substantially perpendicular along a curvature 1320b of the arm 1305b. For example, the arm 1305b may have a curvature, or arc shape, similar to the arc shape 940 of arms 905a, 905b. It is also envisioned that edges of the projections and grooves 1325a, 1325b may extend at angles relative to the curvature 1320b of the arm 1305b.

The castellations 1325 may differ from the castellations 1310 of FIG. 13A in that the projections and grooves 1325a, 1325b may have a depth d2 greater than a depth d1 of the projections and grooves 1310a, 1310b. Additionally, a length L1 of the projections and grooves 1310a, 1310b may be greater than a length L2 of the projections and grooves 1325a, 1325b.

As shown in FIG. 13C, arm 1305c may include castellations 1330, e.g., a series of projections 1330a and grooves 1330b, on an inner surface 1315c of the arm 1305c. The projections 1330a and grooves 1330b may be similarly shaped and sized so that the castellations 1330 are consistent along the inner surface 1315c of the arm 1305c, although in other embodiments, the projections 1330a may be sized and/or shaped differently from the grooves 1330b, and/or individual projections 1330a and/or grooves 1330b may be shaped and/or sized differently. For example, projections 1330a may be separated a distance L3 along the groove 1330b apart from each other, and have a length L4 and/or L5, different from the length than L3. In some embodiments, a length L4 may be a length of the projection 1330a at its peak 1335 and a length L5 at its base 1340. The projection 1330a may extend a depth d3 from the groove 1330b, which may be different from lengths L3, L4, and/or L5. For example, projections 1330a may be narrower than projections 1310a, 1325a, which may provide increased biting into tissue for gripping to minimize slippage. In some embodiments, edges of the castellations may be filleted, chamfered, and/or rounded to further minimize tissue damage. In some embodiments, the edges of the projections and grooves 1330a, 1330b may be substantially perpendicular along a curvature 1320c of the arm 1305c. For example, the arm 1305c may have a curvature, or arc shape, similar to the arc shape 940 of arms 905a, 905b. It is also envisioned that edges of the projections and grooves 1330a, 1330b may extend at angles relative to the curvature 1320c of the arm 1305c.

As shown in FIG. 13D, arm 1305d may include castellations 1345, e.g., a series of projections 1345a and grooves 1345b, on an inner surface 1315d of the arm 1305d. In some embodiments, the projections 1345a may be sized and/or shaped differently from the grooves 1345b, and/or individual projections 1345a and/or grooves 1345b may be shaped and/or sized differently. It is also envisioned that the projections 1345a and grooves 1345b may be similarly shaped and sized so that the castellations 1345 are consistent along the inner surface 1315d of the arm 1305d. For example, a length L6 of the groove 1345b may be different than a length L7 of the projection 1345a, e.g., in some embodiments length L7 may be greater than length L6, or length L6 may be greater than length L7. For example, the projections 1345a may be trapezoidal in shape for increased tissue biting and grip. The projection 1345a may extend a depth d4 from the groove 1345b, which may be different from lengths L6 and/L7. In some embodiments, edges of the castellations may be filleted, chamfered, and/or rounded to further minimize tissue damage. The projections and grooves 1345a, 1345b having a trapezoidal shape as described may allow for tissue to reflow in the grooves 1345b in high compressive tissue gripping. The arm 1305d may have a curvature, or arc shape, similar to the arc shape 940 of arms 905a, 905b. Edges of the projections and grooves 1345a, 1345b may extend at angles relative to the curvature 1320d of the arm 1305d.

As shown in FIG. 13E, arm 1305e may include castellations 1350, e.g., a series of projections 1350a and grooves 1350b, on an inner surface 1315e of the arm 1305e. In some embodiments, the projections 1350a may be sized and/or shaped differently from the grooves 1350b, and/or individual projections 1350a and/or grooves 1350b may be shaped and/or sized differently. It is also envisioned that the projections 1350a and grooves 1350b may be similarly shaped and sized so that the castellations 1350 are consistent along the inner surface 1315e of the arm 1305e. For example, a length L8 of the groove 1350b may be different than a length L9 of the projection 1350a, e.g., in some embodiments length L9 may be greater than length L8, or length L8 may be greater than length L9. The projection 1350a may extend a depth d5 from the groove 1350b, which may be different from lengths L8 and/L9. In some embodiments, edges of the castellations may be filleted, chamfered, and/or rounded to further minimize tissue damage. The arm 1305e may have a curvature, or arc shape, similar to the arc shape 940 of arms 905a, 905b. It is also envisioned that edges of the projections and grooves 1350a, 1350b may extend at angles relative to the curvature 1320e of the arm 1305e. Additionally, an edge of the projection 1350a may include a curvature 1355, configured so that the projection 1350a may act as a hook, or extension, to better grasp tissue in the corner 1360 to reduce tissue slippage when grasped by the arm 1305e. Additionally, the hook may aide in pulling the tissue in a proximal direction while the arms 1305e are actuated to a closed position.

Referring now to FIGS. 14A-15C, a grasping system may include a handle 1405 disposed at a proximal end of a grasping system 1400 having an end effector 1410 at a distal end of the grasping system 1400. The handle 1405 may be a single-handed handle, so that a medical professional may operate the handle 1405 with one hand while using the other hand for actuating other accessories during the procedure. Exemplary embodiments of the handle 1405 according to the present disclosure are shown, which may include a ratchet mechanism 1415. The ratchet mechanism 1415 may allow the medical profession to close the end effector 1410 at a desired position to grasp and hold tissue for an endoscopic, e.g., gastrojejunostomy, procedure, and to hold the end effector 1410 in a locked, closed position through the duration of the procedure while the medical professional may be free to use their hands for actuating other accessories. Additionally, the medical professional may unlock the ratchet mechanism 1415 to open the end effector 1410 and release tissue. In embodiments, the handle 1405 may allow for tactile feedback to the medical professional so that a desired closing force may be used to grasp and hold the tissue without damaging (e.g., perforating) tissue.

Referring now to FIGS. 14B-14F, a handle 1405b, 1405c, 1405d, 1405e, 1405f, may extend along a longitudinal axis 1402, and may include a ratchet mechanism 1415 and a movable portion 1420. It is understood that the longitudinal axis 1402 may be coaxial to longitudinal axes of the grasping system, e.g., 370, 415, 515, 615, 715, 815, 902. The movable portion 1420 may be movable along the longitudinal axis 1402. For example, a medical professional may adjust the movable portion 1420 in a proximal and/or distal direction by adjusting the actuator 1425, 1426, 1430, and/or 1435. When the actuator 1425, 1426, 1430, and/or 1435 is adjusted, the movable portion may be adjusted along a zipper 1440, extending along the longitudinal axis 1402. As described above, a drive wire may be connected to the movable portion 1420 of the handle 1405b-1405f. In this configuration, the drive wire may be manipulated by the medical profession to actuate an end effector, e.g., extending and/or retracting the end effector from a sheath (see FIG. 11A). In other embodiments, the movable portion 1420 may be connected to a sheath so that the medical profession may actuate the sheath to extend and/or retract over the end effector (see FIG. 11B).

The actuator 1425, 1426, 1430, 1435 may be any configuration that allows a medical professional to adjust the movable portion with one hand along the zipper 1440. As shown in FIG. 14B, the actuator 1425 may be a nut, bolt, pin, screw, or other component extending substantially perpendicular from a surface 1445 of the zipper 1440. The medical professional may be able to raise the actuator 1425 out of contact from the zipper 1440, e.g., by rotating the actuator 1425 and/or lifting the actuator 1425 away from the zipper 1440. When the actuator 1425 and the zipper 1440 are not contacting each other, the movable portion may be adjusted along the zipper to the desired location. In this manner, a coarse tuning may be achieved, in that a general position may be set. The medical professional may then rotate and/or actuate the actuator 1425 back into contact with the zipper 1440. As shown in FIG. 14C, an actuator 1430 may be a knob, or other rotational mechanism, which may allow for fine tuning of the position of the movable portion 1420. In this manner, the medical professional may rotate the actuator 1430, which may include a mechanism to engage with the zipper 1440 and move over individual zipper teeth to achieve a fine-tuned position.

Referring now to FIG. 14D, actuator 1426 may be similar to the actuator 1425, e.g., as a ball detent that may be movable in and out of contact from the zipper 1440. The actuator 1426 may include a housing 1427, for receiving and/or retaining a locking component 1428 and/or a compression component 1429. The housing 1427 may be coupled to the movable portion 1420. In embodiments, the housing 1427 may include a cavity for receiving and/or retaining the locking component 1428 and/or the compression component 1429. The housing 1427 may be configured so that the locking component 1428 is positionable in proximity to the zipper 1440. The locking component 1428 may be engageable with teeth of the zipper 1440 to lock the movable portion 1420 in a desired position. In some embodiments, the locking component 1428 may be formed as a ball, although the locking component 1428 may be formed in any shape to engage and/or disengage from the teeth of the zipper 1440 in a direction substantially perpendicular to the surface 1445 of the zipper 1440.

The compression component 1429 may be included in the actuator 1426 to adjust a force applied to the locking component 1428 against the teeth of the zipper 1400. The compression component 1429 may be disposed between the locking component 1428 and a cap 1431, and may compress and/or expand in response to a user applying a force to the cap 1431. In embodiments, the compression component 1429 may be formed as a helical spring, disposed along axis 1403. The compression component 1429 may be disposed vertically above the locking component 1428 along the axis 1403, and the cap 1431 may be disposed vertically above the compression component 1429.

The cap 1431 may be attachable to the housing 1427 to enclose the locking component 1428 and/or the compression component 1429. In embodiments, a user may apply a force in a direction of arrow 1432 on a cover 1431a of the cap 1431, to compress and/or expand the compression component 1429 so that the locking component 1428 engages and/or disengages with the teeth of the zipper 1440. The compression component 1429 may provide additional force against the locking component 1428, to increase contact and/or frictional forces needed to move the movable element 1420 along the axis 1402. The cap 1431 may additionally and/or alternatively be rotatable about axis 1403 in a direction indicated by arrow 1433, which may lock and/or unlock the locking component 1428 in a desired position. For example, the user may twist the cap 1431 about the axis 1403, which may engage with the housing 1427 to hold the actuator 1426 in a desired vertical position and thus maintaining a position of the movable element 1420.

As shown in FIG. 14E, actuator 1435 may include a clip 1450 and a lever 1455. The clip 1450 may have a spring force so that a medical professional may actuate the lever 1455 to disengage the clip 1450 from the zipper 1440 so that the movable portion 1420 may be adjustable along the zipper 1440. The spring force of the clip 1450 may allow the clip to engage with the zipper 1440 when the lever 1455 is released by the medical professional.

Referring now to FIG. 14F, the handle 1405f may have a resilient tooth 1475, which may be integrally formed with the handle 1405f. The resilient tooth 1475 may be a projection configured to engage and lock with the teeth of the zipper 1440 as the handle 1405f is adjusted along the longitudinal axis 1402. The tooth 1475 may be resilient, e.g., having a flexibility to engage with the zipper 1440. For example, the tooth 1475 may be configured to spring, or flex, in response to moving over the teeth of the zipper 1440 along the longitudinal axis 1402. The tooth 1475 may be bounded by a first slit 1480a and a second slit 1480b to allow for resilient flexing in response to movement over the teeth of the zipper 1440. The first and second slits 1480a, 1480b may be cuts, or openings, to define a partial separation of the tooth 1475 from the handle 1405f. The first and second slits 1480a, 1480b may extend substantially parallel along the longitudinal axis 1402, and/or substantially perpendicular to the protrusion of the tooth 1475. This partial separation of the tooth 1475 from the handle 1402e provides a “diving board” configuration so that the tooth 1475 may flex to engage with the teeth of the zipper 1440.

Referring now to FIG. 14G, a handle 1405g may rotate an end effector. For example, an actuator 1460 may be actuated by a medical professional along the longitudinal axis 1402, for turning rotatable portion 1465 about the longitudinal axis 1402 as indicated by arrow 1470. For example, an end effector may be fixed while a sheath is movable by a medical professional in a distal and/or proximal direction (see FIG. 11B).

FIGS. 15A-15C illustrate another exemplary embodiment of a handle 1500 in accordance with the present disclosure. As described above, a drive wire DW may be connected to a movable portion 1520 of the handle 1500 and movable relative to a stationary portion 1515. For example, a proximal end of a drive wire DW may be connected at a joint 1510 to the movable portion 1520 by a crimp bolt 1505. The movable portion 1520 may be movable by actuator 1425, 1430, 1435 by the medical professional, along the longitudinal axis 1502. Additionally, a rotator knob 1525 may be disposed along the longitudinal axis 1502 so that the drive wire DW extends through the rotator knob 1525. The rotator knob 1525 may be rotated about the drive wire DW and longitudinal axis 1502 by the medical professional, for rotation of the drive wire DW and, consequently, an end effector attached at a distal end of the drive wire DW.

As shown in FIG. 15C, the rotator knob 1525 is illustrated in an exploded view. As described above, the drive wire DW may extend through the rotator knob 1525. A washer 1530, e.g., a friction washer, may be disposed between the rotator knob 1525 and a clamp 1535, so that the medical professional may have additional control over rotation. The clamp 1535 may be connectable to the handle 1500 at a distal end of the handle 1500, where the sheath may be connected to the handle 1500. In some embodiments, the sheath may be heat shrunk at the proximal end for strain relief.

It is understood that the exemplary embodiments of handles illustrated in FIGS. 14A-15C may be utilized in endoscopic gastrointestinal (e.g., gastrojejunostomy) procedures for grasping and holding tissue. Additionally, the handles may be utilized with other types of devices, including but not limited to forceps, pulmonary devices, urology devices, and/or cardiovascular devices.

Referring now to FIGS. 16A-16F, schematics of an exemplary embodiment of an endoscopic procedure, e.g., a gastrojejunostomy, in accordance with the present disclosure, are shown. For example, as shown in FIG. 16A, in a first step 1600 a nasocatheter 1630 may be inserted into a patient, e.g., through the nose and esophagus into a stomach and into a small bowel or jejunum, as described above with respect to FIGS. 3-8C. A distal end 1635 of the nasocatheter may be positioned a desired distance in the jejunum as determined by the medical professional. The nasocatheter may be allowed to migrate some portion of the way into position, e.g., by peristaltic motion acting upon projections on the exterior of the catheter. At step 1605, an endoscope 1640 may be inserted into a patient's stomach, so that a distal end 1645 is positioned in the stomach at a region near the distal end 1635 of the nasocatheter 1630 in the small bowel.

When the distal end 1645 of the endoscope 1640 is in the desired position, FIG. 16C shows step 1610, where the stomach wall may be perforated so that an accessory may be extended to tissue of the small bowel in the region of the distal end 1635 of the nasocatheter 1630. For example, a grasping system having an end effector 1650 at a distal end as described above with respect to FIGS. 9A-13E may be extended out of the endoscope 1640. The end effector 1650 may grasp and hold tissue of the small bowel at a position indicated by the nasocatheter 1630, so that an anastomosis may be formed in a desired position. For example, a light source emission at a distal end of the nasocatheter 230 may mark a position and guide a medical professional to make an incision at the marked position in the small bowel.

Referring now to FIG. 16D, at step 1615, a needle 1655 or other perforation mechanism may pierce the small bowel, and a guidewire may hold the position. The small bowel tissue may continue to be held by end effector 1650 so the medical professional may be able to place an anastomosis device (e.g., implant) in the desired position. FIG. 16E shows that at step 1620, an anastomosis stent 1660 may be deployed from the nasocatheter 1630. The stent 1660 may join the patient's stomach to the small bowel, creating a bypass. For example, a delivery device may be inserted through the endoscope, possibly over the guidewire, through the opening in the stomach wall and opening in the jejunum, a distal retention member on the stent deployed inside the jejunum, the stent and delivery may then be retracted proximally to appose the small bowel against the stomach wall, at which point a proximal retention member on the stent may be deployed within the stomach to anchor the stent across the openings and create a bypass conduit for stomach content to flow through. At step 1625 illustrated at FIG. 16F, a pyloric occlusion 1665 may be deployed by the endoscope 1640. The pyloric occlusion 1665 may be any configuration to prevent food, liquid, and/or other nutrients from flowing from the stomach into the duodenum, and/or as described in currently pending application filed concurrently, entitled “Devices, Systems, and Methods for Pyloric Occlusion,” (Attorney Docket No. 8150.0517), which is herein incorporated by reference in its entirety. For example, food, liquid, and/or nutrients may flow from the stomach into the small bowel via the stent 1660, bypassing the duodenum. This may prevent absorption in the patient's digestive tract, promoting weight loss and reducing risk of type-2 diabetes. When the pyloric occlusion is deployed, the medical professional may remove the nasocatheter 1630 and the endoscope 1640.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the subject matter of the claims.

What is claimed is:

1. A method for performing an endoscopic procedure on a patient, the method comprising:
inserting a catheter in the patient, a distal end of a catheter being selectively positioned in a small bowel of the patient;
inserting an endoscope in the patient, a distal end of the endoscope being selectively positioned in a stomach of the patient based on the selected position of the catheter in the small bowel; and
actuating a grasping system to extend an end effector for grasping selected tissue of the small bowel at the selected position of the catheter;
wherein the end effector includes a first arm and a second arm extending from a connection at a proximal end of the first and second arms and movable between an open position and a closed position, wherein the first arm and the second arm are arc shaped and form arcuate shaped tissue-engaging surfaces extending proximally from respective distal tips thereof and facing each other such that the first arm and the second arm are configured for atraumatically closing around the small bowel as the first and second arms are actuated to a closed position to enclose the small bowel atraumatically between the arcuate-shaped tissue-engaging surfaces proximal to the distal tips of the first arm and the second arm.

2. The method according to claim 1, wherein the catheter is selectively positioned at a first location in the small bowel and the endoscope is selectively positioned at a second location apposing the first location.

3. The method according to claim 1, further comprising:
creating an opening in the stomach;
grasping the tissue of the small bowel with the grasping system through the stomach opening;
creating an opening in the small bowel; and
delivering an anastomotic device across the openings;
wherein the device apposes the stomach and small bowel at the respective selected positions and creates a conduit for stomach content to flow therethrough.

4. The method according to claim 1, wherein the catheter includes projections that are acted upon by peristaltic motion to propel the catheter to the selected position in the small bowel.

5. The method according to claim 1, wherein the end effector is actuatable relative to a sheath, such that the first and second arms are actuated between an unconstrained open position in response to the end effector extending out of the sheath, and a constrained closed position in response to the end effector retracting within the sheath.

6. A method for performing an endoscopic procedure on a patient, the method comprising:
inserting a catheter in the patient, a distal end of a catheter being selectively positioned in a small bowel of the patient;
inserting an endoscope in the patient, a distal end of the endoscope being selectively positioned in a stomach of the patient based on the selected position of the catheter in the small bowel; and
actuating a grasping system to extend an end effector for grasping selected tissue of the small bowel at the selected position of the catheter, the end effector having a first arm and a second arm extending from a proximal end of the first and second arms to respective distal tips, and movable between an open position and a closed position;
causing the first arm and the second arm to move together to the closed position to atraumatically encompass a portion of the small bowel with only the distal tips of the first arm and the second arm contacting each other, and without pinching the small bowel between the first arm and the second arm; and
moving the portion of the small bowel encompassed and grasped between the first arm and the second arm proximally.

7. The method according to claim 6, wherein the catheter is selectively positioned at a first location in the small bowel and the endoscope is selectively positioned at a second location apposing the first location.

8. The method according to claim 6, further comprising:
creating an opening in the stomach;
grasping the tissue of the small bowel with the grasping system through the stomach opening;
creating an opening in the small bowel; and
delivering an anastomotic device across the openings;
wherein the device apposes the stomach and small bowel at the respective selected positions and creates a conduit for stomach content to flow therethrough.

9. The method according to claim 6, wherein the catheter includes projections that are acted upon by peristaltic motion to propel the catheter to the selected position in the small bowel.

10. The method according to claim 6, wherein the end effector is actuatable relative to a sheath, such that the first and second arms are actuated between an unconstrained open position in response to the end effector extending out of the sheath, and a constrained closed position in response to the end effector retracting within the sheath.

11. The method according to claim 6, further comprising pulling the portion of the small bowel encompassed and grasped between the first arm and the second arm relative to portions of the small bowel on either side of and not encompassed by the first arm and the second arm.

12. The method of claim 6, further comprising actuating the grasping system by moving the first arm and the second arm between a closed position, at least partially drawn into a flexible sheath, and an open position, outside the flexible sheath.

13. The method of claim 12, further comprising navigating the flexible sheath into a natural orifice of the patient, into the stomach of the patient, and through an opening created in the stomach to grasp the small bowel of the patient.

14. A method for performing an endoscopic procedure on a patient, the method comprising:
navigating a flexible sheath into a natural orifice of the patient, into the stomach of the patient, and through an opening created in the stomach to grasp the small bowel of the patient;
actuating a grasping system to extend an end effector to grasp a selected portion of the small bowel between a first arm and a second arm of the end effector, the first arm and the second arm extending from a proximal end of the first and second arms to respective distal tips, and movable between an open position, in which the selected portion of the small bowel may be positioned between the first arm and the second arm, and a closed position;
causing the first arm and the second arm to move together from the open position to the closed position to atraumatically encompass the selected portion of the small bowel between arcuate surfaces of the first arm and the second arm, and with the distal tips of the first arm and the second arm contacting each other distal to the selected portion of the small bowel, and without pinching the small bowel between the first arm and the second arm; and
moving the selected portion of the small bowel encompassed and grasped between the first arm and the second arm.

15. The method according to claim 14, further comprising extending the flexible sheath through an endoscope and into the patient's stomach.

16. The method according to claim 14, further comprising:
creating an opening in the stomach;
grasping the tissue of the small bowel with the grasping system through the stomach opening;
creating an opening in the small bowel; and
delivering an anastomotic device across the openings;
wherein the device apposes the stomach and small bowel at the respective selected positions and creates a conduit for stomach content to flow therethrough.

17. The method according to claim 14, further comprising actuating the end effector by extending the end effector between a closed position within the flexible sheath and an open position distally outside the flexible sheath.

18. The method of claim 17, further comprising navigating the end effector into the patient in a withdrawn constrained position within the flexible sheath.

19. The method according to claim 14, further comprising pulling the selected portion of the small bowel encompassed and grasped between the first arm and the second arm relative to portions of the small bowel on either side of and not encompassed by the first arm and the second arm.

20. The method of claim 14, further comprising pulling the selected portion of the small bowel towards the stomach to create an anastomosis between the stomach and the selected portion of the small bowel.
